(19) United States
(12) Patent Application Publication
(10) Pub. No.: US 2018/0125530 A1
(43) Pub. Date: May 10, 2018

(54) NATURAL ORIFICE ACCESS DEVICE

(71) Applicant: Applied Medical Resources Corporation, Rancho Santa Margarita, CA (US)

(72) Inventors: Kevin K. Dang, Garden Grove, CA (US); Jeremy J. Albrecht, Rancho Santa Margarita, CA (US); Blaze Brown, Rancho Santa Margarita, CA (US); Adam Hoke, Shellbyville, MI (US); Jacob J. Filek, Rancho Santa Margarita, CA (US); Gary M. Johnson, Rancho Santa Margarita, CA (US); Adeeb Saiduddin, Irvine, CA (US); Alexander Sheehan, Rancho Santa Margarita, CA (US); Alexandra Do, San Clemente, CA (US)

(21) Appl. No.: 15/864,332

(22) Filed: Jan. 8, 2018

Related U.S. Application Data
(62) Division of application No. 14/491,498, filed on Sep. 19, 2014, now abandoned.
(60) Provisional application No. 61/880,641, filed on Sep. 20, 2013.

(51) Int. Cl. A61B 17/34

(52) U.S. Cl. CPC A61B 17/3423; A61B 17/3431; A61B 17/3421; A61B 2017/3452; A61B 2017/3466; A61B 2017/3492; A61B 2017/3443

(57) ABSTRACT

Embodiments of a natural orifice access device that comprises a cut-out portion to provide access to a surgical site and that includes an obturator adapted to interface with the cut-out potion to provide a smooth and consistent contact surface during insertion into the natural orifice.

### CROSS REFERENCE TO RELATED APPLICATIONS

This application claims the benefit of U.S. application Ser. No. 14/491,498, filed Sep. 19, 2014, and U.S. Provisional Application Ser. No. 61/880,641 entitled “Natural Orifice Surgery System” filed Sep. 20, 2013. The above-referenced applications are hereby incorporated by reference in its entirety.

### BACKGROUND

### Technical Field

This application is generally directed to surgical devices, and more particularly, to an adjustable access device adapted for use with a cap, that is useful in natural orifice single-port surgical procedures and that allows surgeons to easily access lesions of varying size and depth within the natural orifice.

### Description of the Related Art

Access devices are commonly used in surgery to facilitate the introduction of various surgical instruments into natural biological vessels, conduits, orifices, cavities, and other interior regions of the body. These access devices include, for example, devices that facilitate the introduction of a needle into a vessel, and trocars that facilitate the introduction of laparoscopic instruments into the abdomen of the body.

Some of these access devices are introduced into regions that include a fluid or gas under pressure. In the case of a needle access device, the pressure may be from a liquid, such as blood. In the case of a trocar, the pressure may be from a gas, such as an insufflation gas. In either case, it is desirable to provide for the introduction of the surgical instrument into the cavity without permitting the escape of the pressurized fluid or gas.

In the case of trocars, a cannula at the distal end of the trocar is typically connected to a seal housing at the proximal end of the trocar. Together the cannula and housing form a working channel through which various instruments can be inserted to access the cavity. Seal mechanisms are commonly disposed in the housing and include a septum valve that seals the working channel when an instrument is in place, and a zero closure valve that seals the working channel when the instrument is removed.

Current surgical access ports allow for single instrument access through each port, or allow for multiple instrument access through a rigid cannula. Some devices, such as transanal endoscopic microsurgery (TEMS) units require that the device be attached to the surgical table to support the weight of the device, as well as to locate the position of the device respective to the patient. These devices do not provide flexibility to the surgeon in selecting instrument size, and they restrict instrument movement with their rigid cannulas. Moreover, access devices having a fixed length may limit a surgeon's ability to access all regions of the natural orifice and may not anchor properly in patients having a higher body mass index (BMI).

Additionally, surgeons are performing laparoscopic surgical procedures through a single or a limited number of access ports. The procedures may be performed through a single two (2) centimeter incision at the umbilicus, trans-vaginally or trans-anally. What is needed is a system that meets the needs of these new procedures, facilitating more flexible movement of laparoscopic instruments through a single or limited number of ports while preventing the escape of pressured fluids or gasses and permitting large specimen removal and that provides access to a greater portion of the natural orifice while anchoring properly in patients having higher BMIs.

### SUMMARY OF THE INVENTION

The invention is directed to a surgical access device that is adapted for retracting and providing access to a surgical site and that is particularly useful in natural orifice surgery. The access device comprises an outer ring, wherein the outer ring is configured to be disposed proximate the natural orifice of the patient and substantially surround the orifice; a tubular body having a proximal end, a distal end, a longitudinal axis and a cut-out portion disposed through a side of the tubular body; a funnel segment extending between and coupling the outer ring and the proximal end of the tubular body, wherein the funnel segment comprises an inner surface that can provide a bearing surface for an obturator used to advance to the retractor into a body cavity and provides a diametric reduction between the relatively large diameter of the outer ring and the relatively smaller diameter of the tubular body, which is sized to fit within a natural orifice with minimal distention of the orifice; and an obturator comprising a proximal bearing surface sized and configured to bear against the inner surface of the funnel segment, a distal dilation surface sized and configured to expand the natural orifice, a straight shaft piece disposed between the distal dilation surface and the proximal bearing surface, and a raised flange disposed on the straight shaft piece, adapted to interface with the cut-out potion to provide a smooth and consistent contact surface during insertion

### BRIEF DESCRIPTION OF THE DRAWINGS

FIG. 1 is a side view of a patient in surgery illustrating an embodiment of the access device positioned on the abdomen and in use.

FIG. 2 is a cross-sectional side view illustrating an embodiment of the access device for use in vaginal procedures.

FIG. 3 is a front view illustrating an embodiment of the access device deployed and in use at the mouth of the patient.

FIG. 4 is a top view illustrated a patient in the prone position with an embodiment of the access device deployed and in use at the anus of the patient.

FIG. 5 is a perspective view of an embodiment of an access device with a cap.

FIG. 6A is a side view of an embodiment of a natural orifice access device. FIG. 6B is a top view of the natural orifice access device of FIG. 6A. FIG. 6C is a partial cut away of the natural orifice access device of FIG. 6A.

FIG. 6D is a side view of another embodiment of a natural orifice access device. FIG. 6E is a top view of the natural orifice access device of FIG. 6D. FIG. 6F is a perspective view of the natural orifice access device of FIG. 6A.

FIG. 6G is a perspective view of an obturator adapted to facilitate introduction of a natural orifice access device into a body orifice such as an anus. FIG. 6H is a side view of the obturator of FIG. 6G.

FIG. 6I is a perspective view of an obturator having a straight shaft piece, adapted to facilitate introduction of a natural orifice access device into a body orifice such as an anus. FIG. 6J is a perspective view of an access device disposed on the obturator of FIG. 6I.

FIG. 7A is a partial side cross section of the natural orifice access device of FIG. 6A with a gel cap coupled therewith.

FIG. 7B is a side cross section of the natural orifice access device of FIG. 6D.

FIG. 7C is a perspective view of a natural orifice access device formed from sections and having cut-out portions or windows in the tubular body of the access device. FIG. 7D is a cutaway view of the access device of FIG. 7C showing the slidable engagement of the sections. FIG. 7E is a cutaway view of the access device of FIG. 7C showing the snap-lock mechanism securing the sections together.

FIG. 7F is a perspective view and a side view of an alternative embodiment of an access device having cut-out portions or windows in the tubular body of the access device.

FIG. 7G is a perspective view of an alternative embodiment of an access device having an inflatable member. FIG. 7H shows a close-up view of the inflatable member. FIG. 7I is a top-down perspective view of the access device showing the check valve port of inflating the inflatable member. FIG. 7J is a cutaway side view showing the check valve and channel disposed in the tubular body of the access device. FIG. 7K is a side view of an access device showing the channel disposed between the check valve and the inflatable member.

FIG. 7L is a perspective view of an obturator, modified with an indent to provide clearance for the inflation port shown in FIGS. 7J and 7K and adapted to facilitate introduction of a natural orifice access device into a body orifice such as an anus.

FIG. 7M is a perspective view and a side view of a perforated natural orifice access device.

FIG. 8A is a side view of the natural orifice access device of FIG. 7A.

FIG. 8B is a top view of the natural orifice access device illustrated in FIG. 7A. FIG. 8C is a perspective view of the natural orifice access device illustrated in FIG. 7A.

FIG. 8D is a perspective view of the natural orifice access device of FIG. 6D with a gel cap.

FIG. 9A is a perspective view of an embodiment of a natural orifice access device including a cap having a plurality of trocars extending there through. FIG. 9B is a perspective view of another embodiment of a natural orifice access device including a cap having a plurality of trocars extending there through.

FIG. 9C is an exploded view of an embodiment of a trocar access device and optional obturator, which is a component of some embodiments of the access device system.

FIG. 10 is a perspective view of an access device disposed through a clamping device.

FIG. 11A is an exploded view of an embodiment of a natural orifice access device having a tubular body formed from threaded sections. FIG. 11B shows the tapered tip of the embodiment of FIG. 11A.

FIG. 12 is an exploded view of an embodiment of a natural orifice access device having a tubular body formed from sections connected by a twist-lock mechanism.

FIG. 13 is a close-up view of the twist-lock mechanism of the embodiment of FIG. 12.

FIG. 14 is a perspective view of an embodiment of a natural orifice access device having a collapsible tubular body.

FIG. 15A is an exploded side view of an embodiment of a natural orifice access device comprising a segmented channel. FIG. 15B is a side view showing the segmented channel disposed through the access device of FIG. 15A.

FIG. 16A is a side view of an embodiment of a natural orifice access device having an adjustable channel length provided by a thread design. FIG. 16B is a side view of the embodiment of FIG. 16A with the channel length adjusted. FIG. 16C is a perspective view of the embodiment of FIG. 16A, showing the helical thread detail.

FIG. 17A is a side view of an embodiment of a natural orifice access device having an adjustable channel length provided by a snap button design. FIG. 17B is a side view of the embodiment of FIG. 17A with the channel length adjusted. FIG. 17C is a side view of the embodiment of FIG. 17B, showing the snap pin detail.

FIG. 18A is a side view of an embodiment of a natural orifice access device having an adjustable channel length provided by a cuff pin design. FIG. 18B is a side view of the embodiment of FIG. 18A with the channel length adjusted. FIG. 18C is a side view of the embodiment of FIG. 18A, showing the slider pin detail.

FIG. 19A is a side view of an embodiment of a natural orifice access device having an adjustable channel length provided by a snap button design. FIG. 19B is a side view of the embodiment of FIG. 19A with the channel length adjusted. FIG. 19C is a perspective view of the embodiment of FIG. 19A, with excess material removed to show the tab detail.

FIG. 20 is a perspective view of an obturator adapted for use with a natural orifice access device having a cut-out portion or window.

Similar components have similar reference numbers throughout.

### DETAILED DESCRIPTION OF CERTAIN EMBODIMENTS

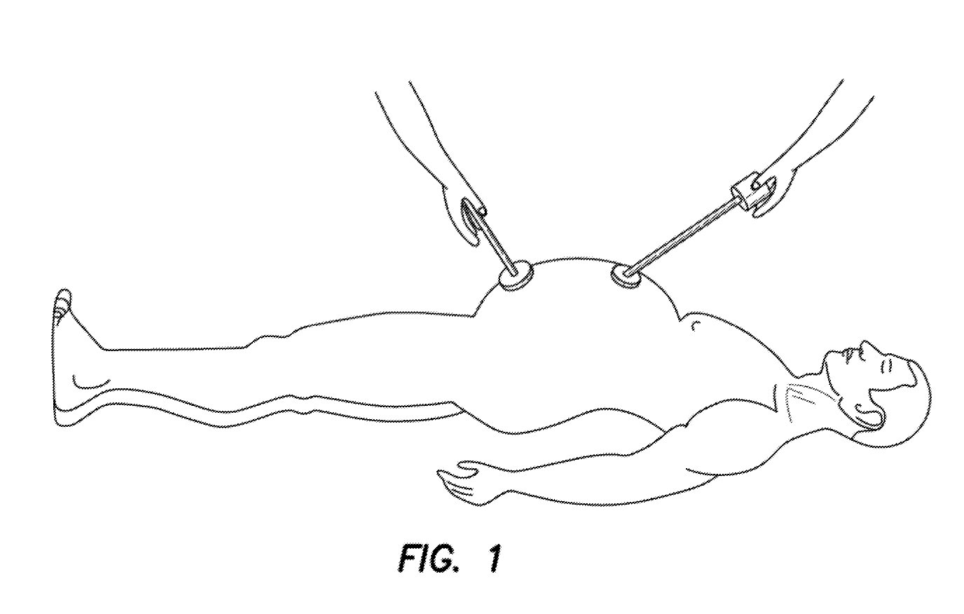

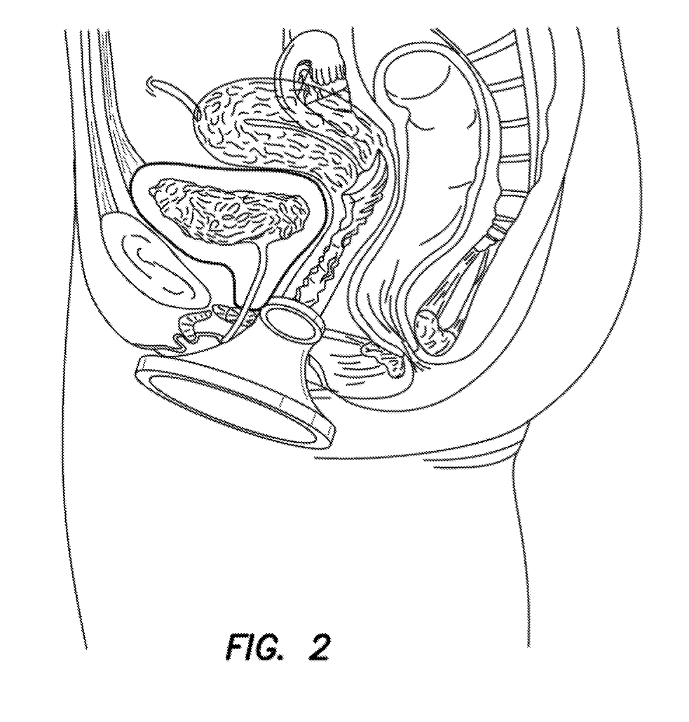

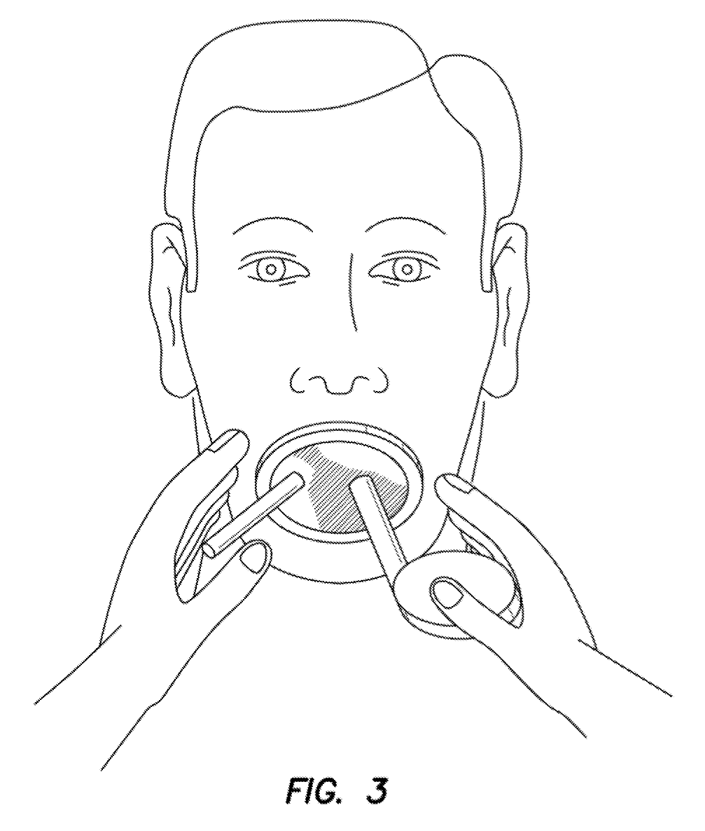

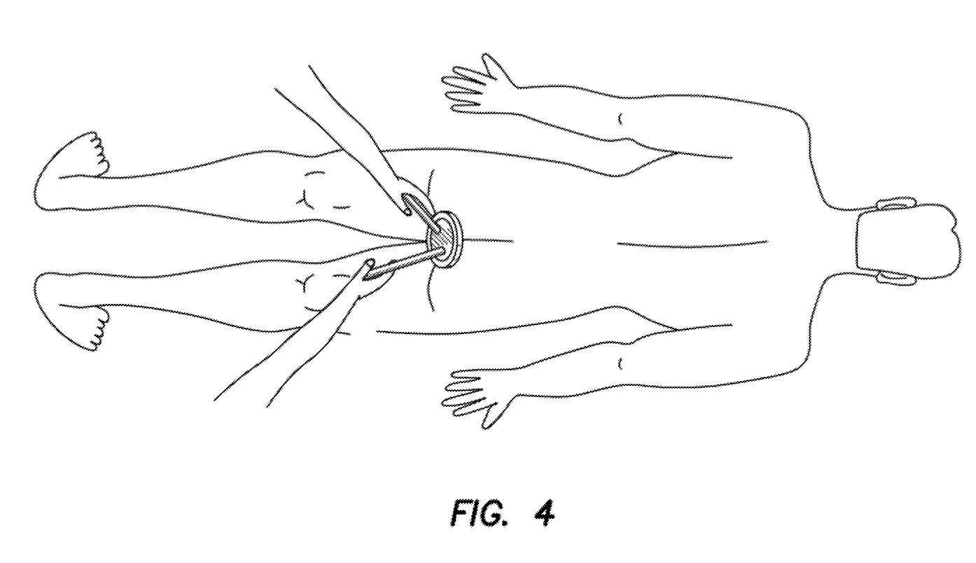

Embodiments of a surgical instrument access device system are useful, for example, for single incision, single port, and/or limited port laparoscopic surgical procedures, for example, abdominal (FIG. 1), transvaginal (FIG. 2), transoral (FIG. 3), and transanal (FIG. 4) procedures. Various surgical instrument access devices are described in U.S. Patent Application Publication No. 2009/0187079, entitled “SURGICAL INSTRUMENT ACCESS DEVICE,” filed Jan. 22, 2009, and U.S. Pat. No. 7,727,146, entitled “WOUND RETRACTOR WITH GEL CAP,” both of which are incorporated by reference in their entireties herein.

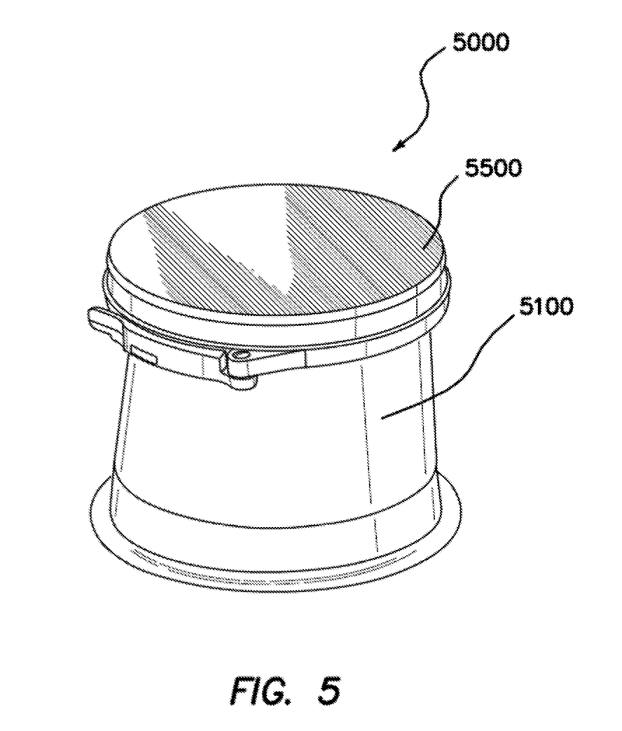

FIG. 5 illustrates a perspective view of an embodiment of an access device system 5000 comprising an access device 5100 and a cap 5500, which is useful in single port and/or limited port procedures. The access device 5100 is placed and/or positioned into, across, and/or through a surgical incision and/or body orifice to enlarge, reshape, and/or isolate the incision or body orifice. The cap 5500 provides an artificial body wall through which instruments access the interior of a patient's body, for example, a body cavity. The components of the access device 5000 comprise any suitable biologically compatible materials.

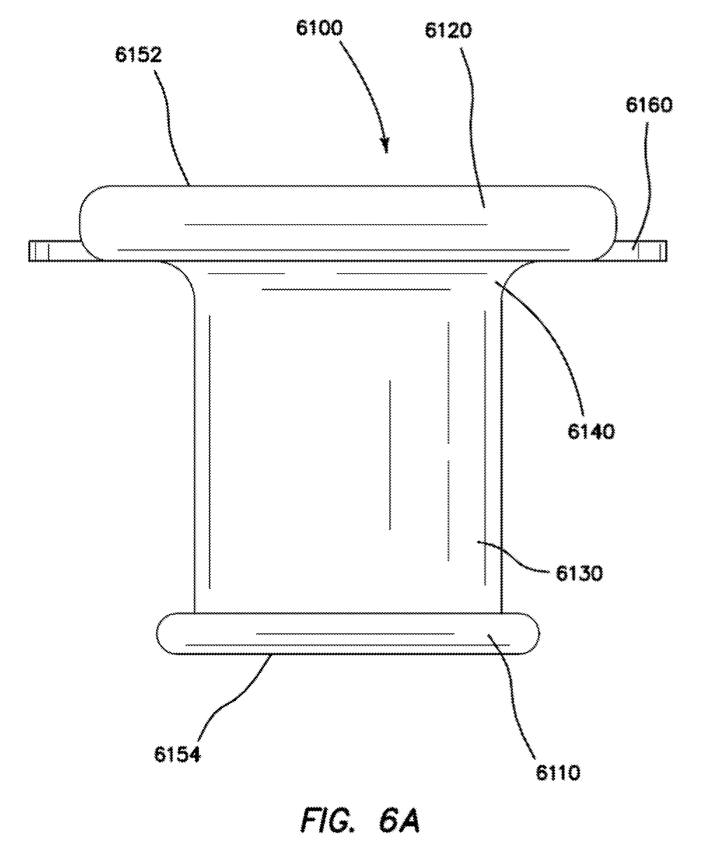

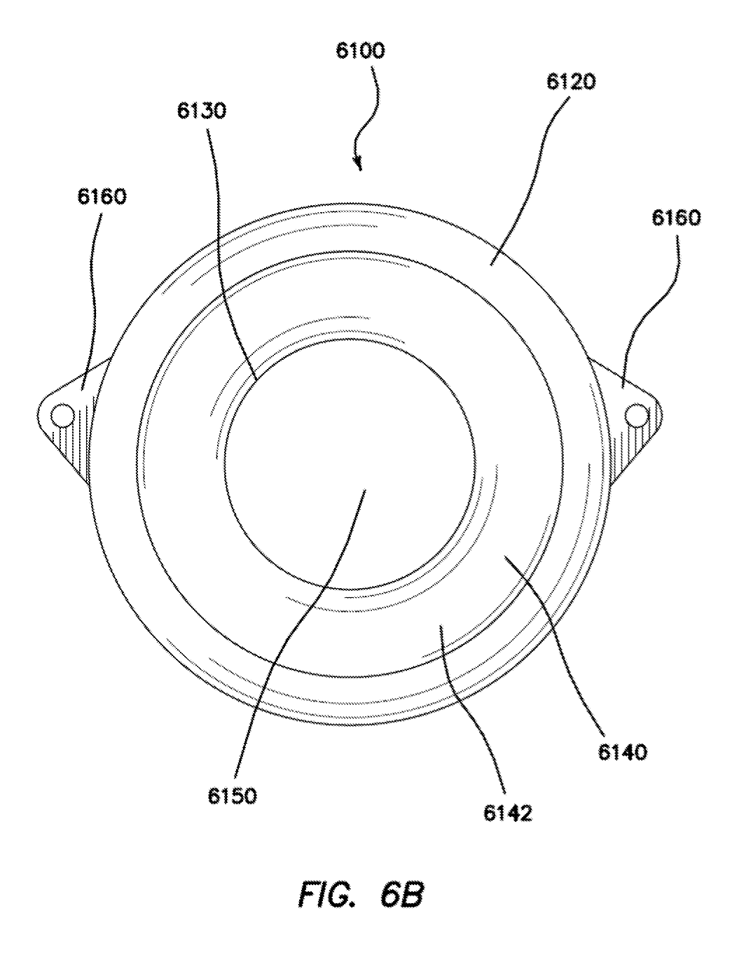

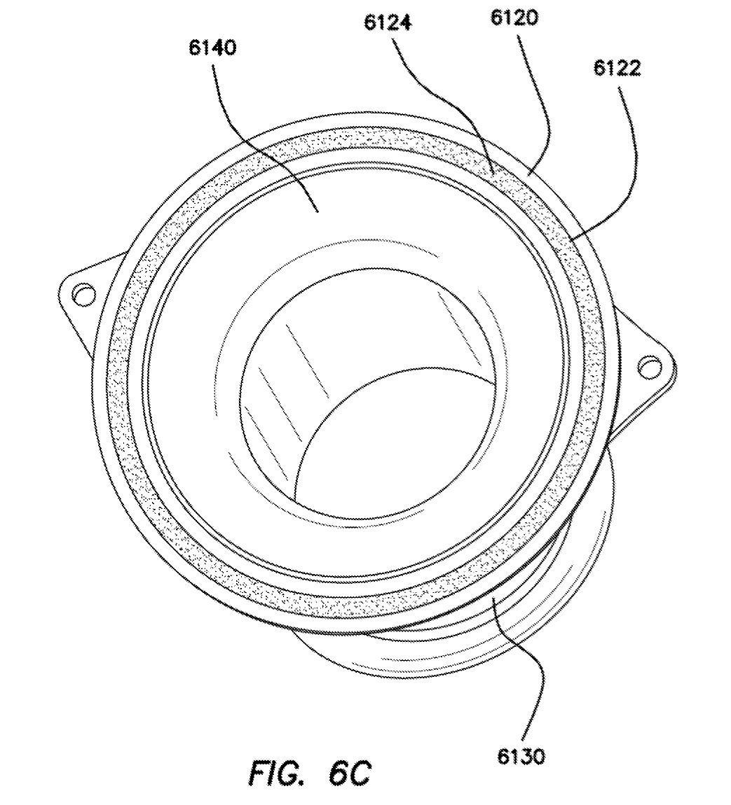

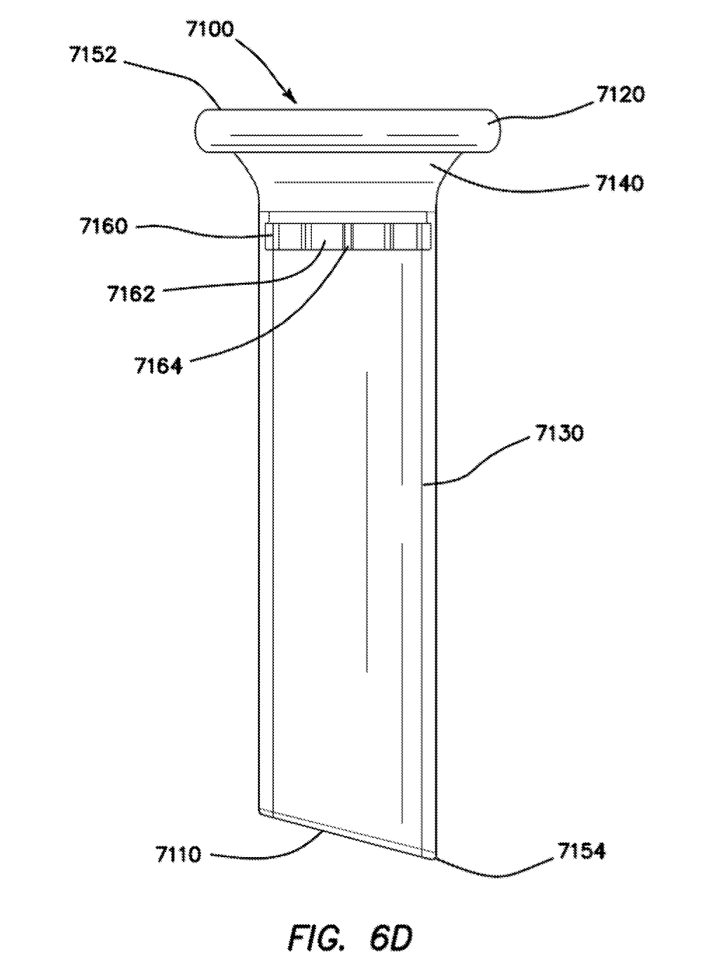

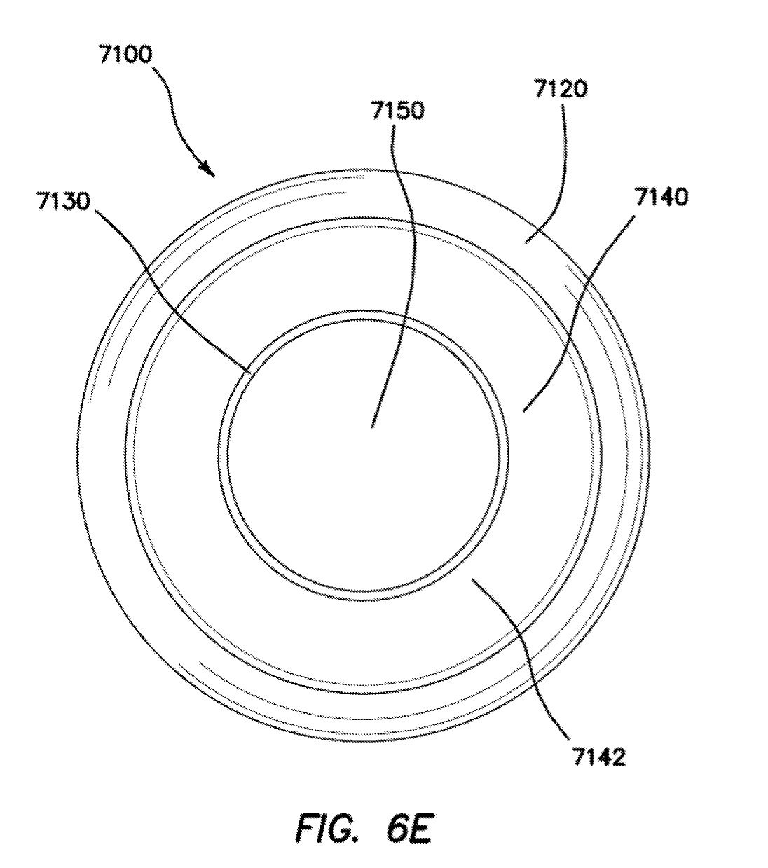

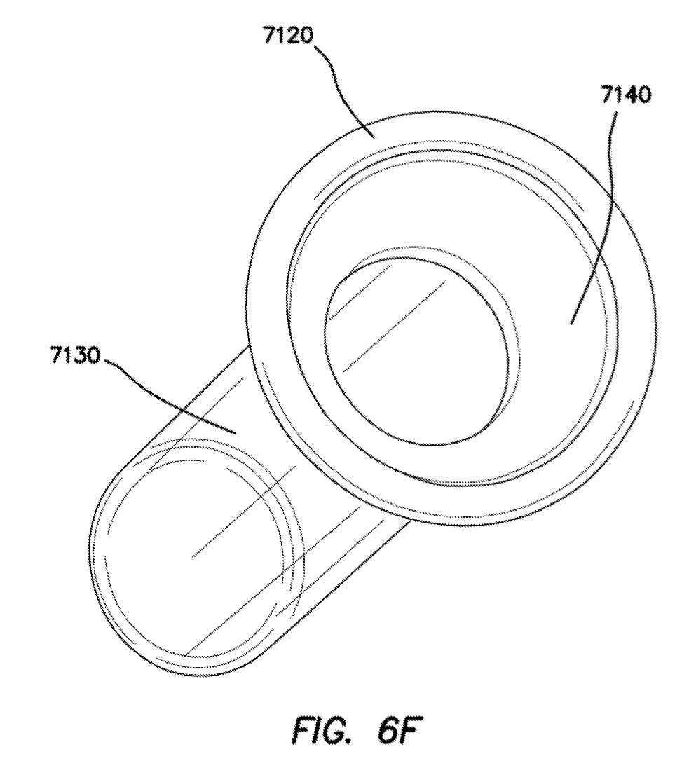

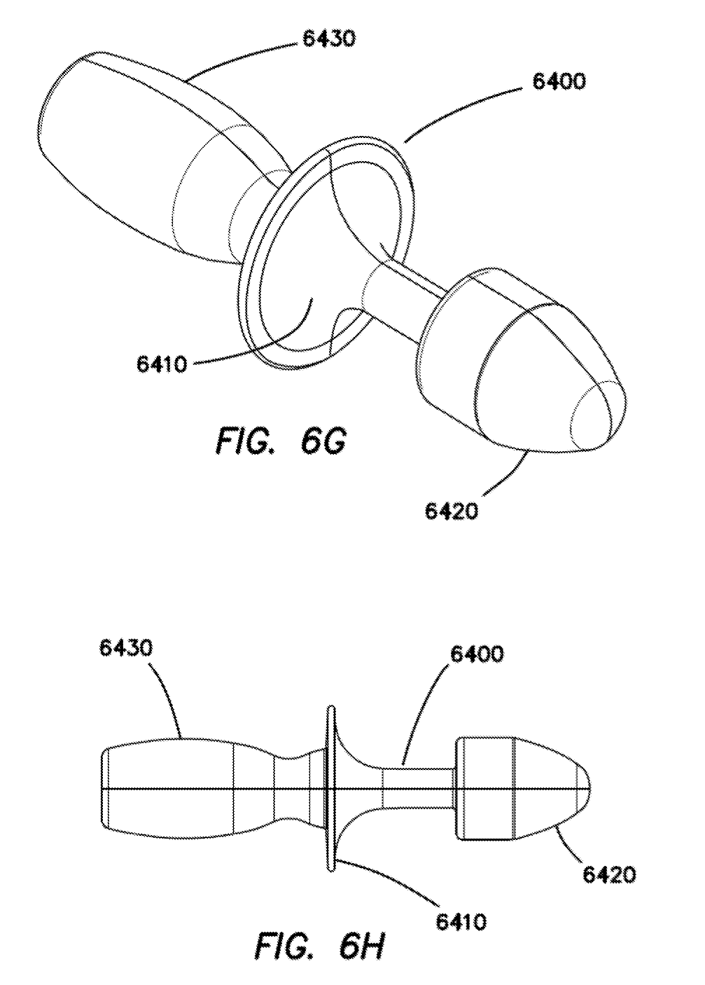

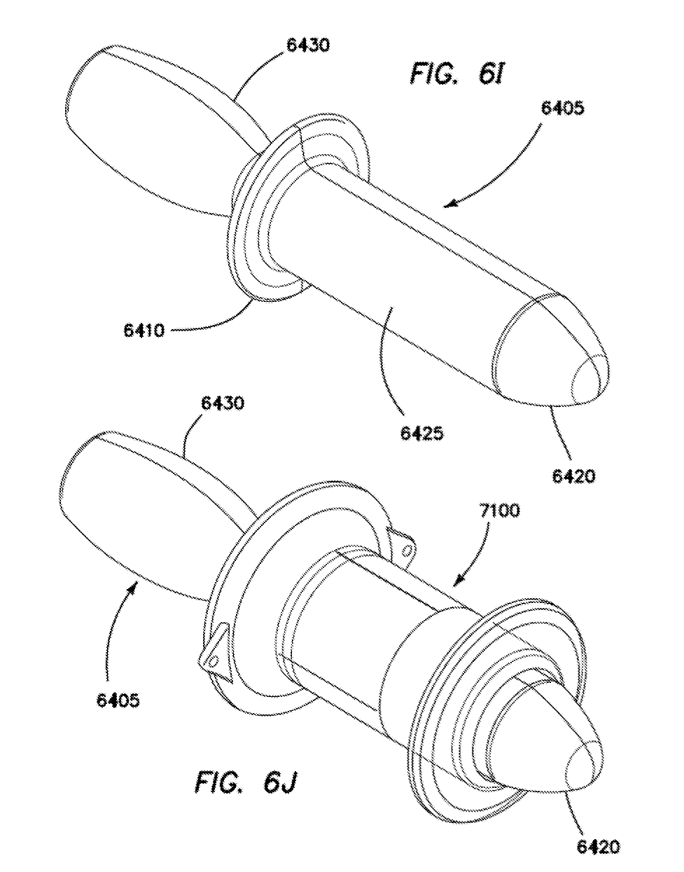

Two embodiments of natural orifice access devices 6100, 7100 sharing certain similarities are illustrated in FIGS. 6-9. One embodiment of access device 6100 is illustrated in FIGS. 6A-6C, 7A, 8A-8C, and 9A. Another embodiment of access device 7100 is illustrated in FIGS. 6D-6F, 7B, 8D, and 9B

The embodiment of the natural orifice access device 6100 illustrated in a side view in FIG. 6A can be adapted for use in a transanal surgical procedure. The access device 6100 comprises an inner or distal ring 6110, an outer or proximal ring 6120, a tubular body 6130, and a funnel segment 6140 extending between and coupling the inner ring 6110 and the outer ring 6120. The tubular body 6130 comprises a relatively flexible material such as a KRATON® polymer or a silicone rubber material, which is substantially cylindrical in the illustrated embodiment. In other embodiments, the tubular body 6130 has another shape, for example, an oval cross section. Some embodiments of the tubular body 6130 comprise one or more coatings that provide additional functionality, for example, an anti-microbial coating.

Embodiments of the inner ring 6110 are sufficiently flexible and compliant to be compressed and/or deformed for insertion into a body orifice such as a patient's anus during a transanal surgical procedure. When subsequently released within an associated body cavity, the inner ring 6110 substantially returns to its original shape or footprint. In some embodiments, the inner ring 6110 assumes a substantially circular shape in a relaxed state, for example, when released within a body cavity. In other embodiments, the inner ring 6110 has another shape in the relaxed state, for example, an oval. The inner ring 6110 assumes a different shape when compressed for insertion through an incision or body orifice, for example, a substantially oval shape, a generally linear shape, a tear-drop shape, or another suitable shape. Those skilled in the art will recognize that in other embodiments, the inner ring 6110 in the relaxed state has a shape other than round, for example, oval, elliptical, or D-shaped. In other embodiments, the inner ring 6110 is substantially rigid, that is, non-compliant under the ordinary conditions under which it is used. In some embodiments, the inner ring extends outward from the surface of the tubular body, as shown, for example, in FIG. 6A, to thereby aid in retaining the access device in the body cavity after it is deployed.

Embodiments of the inner ring 6110 can comprise a generally circular cross section. In other embodiments, the inner ring 6110 comprises another cross-sectional shape, for example, at least one of oval or elliptical, tear-drop shaped, and D-shaped. For example, in embodiments illustrated in FIGS. 6D-6F, the inner ring 7110 can have a cross-sectional shape that is substantially flush with the tubular body 7130 of the access device 7100 as further described herein. Those skilled in the art will understand that other cross sections are used in other embodiments. As further discussed herein with respect to the flexion region of the inner ring 6110, some embodiments of the inner ring 6110 comprise at least one notch and/or weak spot, which facilitate folding or deforming the inner ring 6110, thereby facilitating insertion and/or removal of the inner ring 6110.

Returning to FIG. 6A, the outer ring 6120 is proximal the funnel section 6140. In the illustrated embodiment, the outer ring 6120 has a substantially circular footprint. As further discussed herein, the outer ring 6120 can be sized and configured to sealingly couple to a cap or other access device thereon. In some embodiments, one or more suture points 6160 can be disposed on the access device 6110 adjacent the outer ring 6120.

With reference to FIG. 6B, a top view of access device 6100 is illustrated. In the illustrated embodiment, outer ring 6120 has a generally circular profile. Additionally, in the illustrated embodiment, two suture points 6160 are generally diametrically opposed relative to the generally circular profile of the outer ring 6120. In other embodiments, the access device can include more or fewer than two suture points disposed of various locations relative to the outer ring 6120.

With continued reference to FIG. 6B, the tubular body 6130 has a generally circular profile defining a generally cylindrical passage 6150. The generally cylindrical passage 6150 is desirably large enough to accommodate more than one laparoscopic instrument there through such that a single natural orifice access device can be used to provide access for multiple surgical instruments in a body cavity. Moreover, generally cylindrical passage 6150 is desirably large enough such that multiple surgical instruments positioned there through can be translated or pivoted relative to one another, allowing a surgeon to manipulate the instruments as desired during a surgical procedure. The generally cylindrical passage extends between a proximal end 6152 of the access device 6100 adjacent the outer ring 6120 to a distal end 6154 of the access device 6100 adjacent the inner ring 6110 (FIG. 6A).

With continued reference to FIG. 6B, in the illustrated embodiment, the funnel segment 6140 provides a diametric reduction between the relatively large diameter of the outer ring 6120, which is sized and configured to be removably coupled to an access device such as a cap, and the relatively smaller diameter of the passage 6150, which is sized to fit within a natural orifice with minimal distention of the orifice. The funnel segment 6140 has an inner surface 6142 which can provide a bearing surface for an obturator used to advance to the access device 6100 into a body cavity. In some embodiments, the funnel segment 6140 can have a substantially linear taper between the relatively large diameter and the relatively smaller diameter such that the inner surface 6142 is a frusto-conical segment. In other embodiments, the funnel segment 6140 can have a curved profile between the relatively large diameter and the relatively smaller diameter.

In some embodiments, a natural orifice access system can include an access device 6100 and an optional obturator 6400 (FIG. 6G-6H). The obturator can have a proximal bearing surface 6410 sized and configured to bear against the inner surface 6142 of the funnel segment 6140 and a distal dilation surface 6420 sized and configured to expand a natural orifice for passage of the access device 6100. Thus, during insertion of the access device 6100 into a natural orifice, the dilation surface 6420 expands a pathway to a surgical site in a body cavity while the obturator bears on the inner surface 6142 of the funnel segment 6140 to advance the access device 6100 into position in the surgical site. Furthermore, in some embodiments, the obturator can have a handle 6430 at a proximal end thereof adapted to facilitate selective twisting or rotation of the obturator about a longitudinal axis thereof during insertion.

It can be desirable that the outer ring 6120 is relatively stiff compared with the relatively flexible tubular body 6130 of the access device 6100 so that the outer ring 6120 can sealingly engage a device such as a cap. With reference to FIG. 6C, a perspective view of the access device is illustrated with a partial cutaway of the outer ring 6120. In the illustrated embodiment, the outer ring 6120 includes an annular groove 6122 formed therein in which a reinforcing member 6124 is disposed. In some embodiments, the reinforcing member 6124 can comprise a metallic member such as a wire formed into a ring shape. For example, in some embodiments the reinforcing member 6124 can comprise a stainless steel ring positioned within the groove 6122 during manufacture of the access device 6100. In other embodiments, the reinforcing number 6124 can comprise an injectable nonmetallic member. For example, in some embodiments, a glass filled polymer or polycarbonate material can be injected into the groove 6122 during manufacture of the access device 6100.

While the illustrated embodiments of access device 6100 include a reinforcing member to enhance the rigidity of the outer ring 6120, in other embodiments, the access device 6100 can be formed in a multiple-shot molding process. For example, in some embodiments, an inner segment of the access device defined by the tubular body 6130 and the inner ring 6110 is formed in one molding operation from a flexible material, and an outer segment of the access device 6100 defined by the funnel segment 6140 and the outer ring 6120 is formed in another molding operation from a relatively rigid material such as a polycarbonate material or other suitable material. One embodiment of access device 7100 formed in a multiple-shot molding process is illustrated in FIGS. 6D-F, 7B, 8D, and 9B.

With continued reference to FIG. 6C, the illustrated embodiment includes a continuous generally annular groove. In other embodiments, a plurality of noncontiguous recesses can each receive one of a plurality of reinforcing members. Moreover, in some embodiments, the outer ring can include two or more concentric generally annular grooves, which each receive a corresponding reinforcing member.

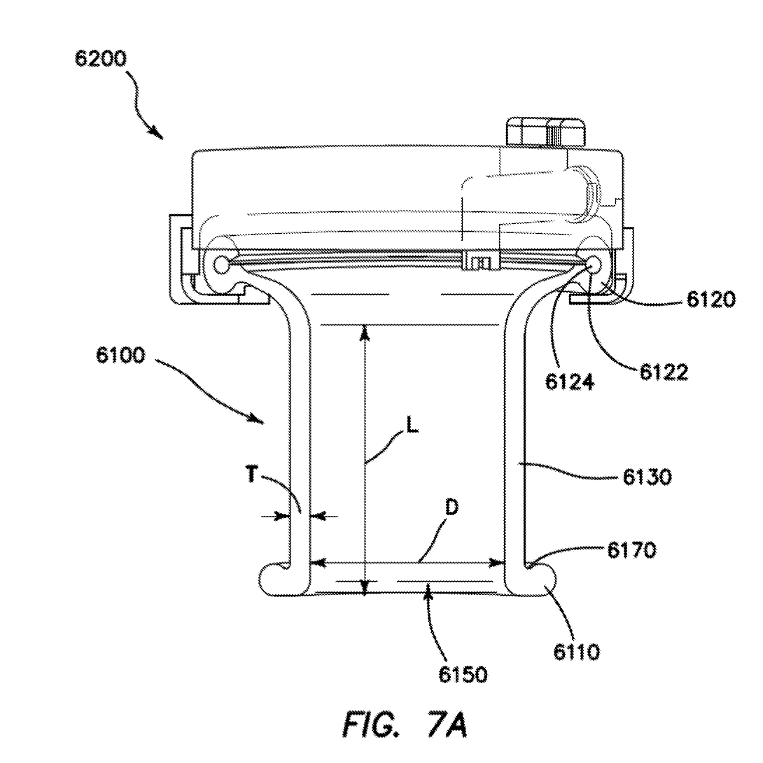

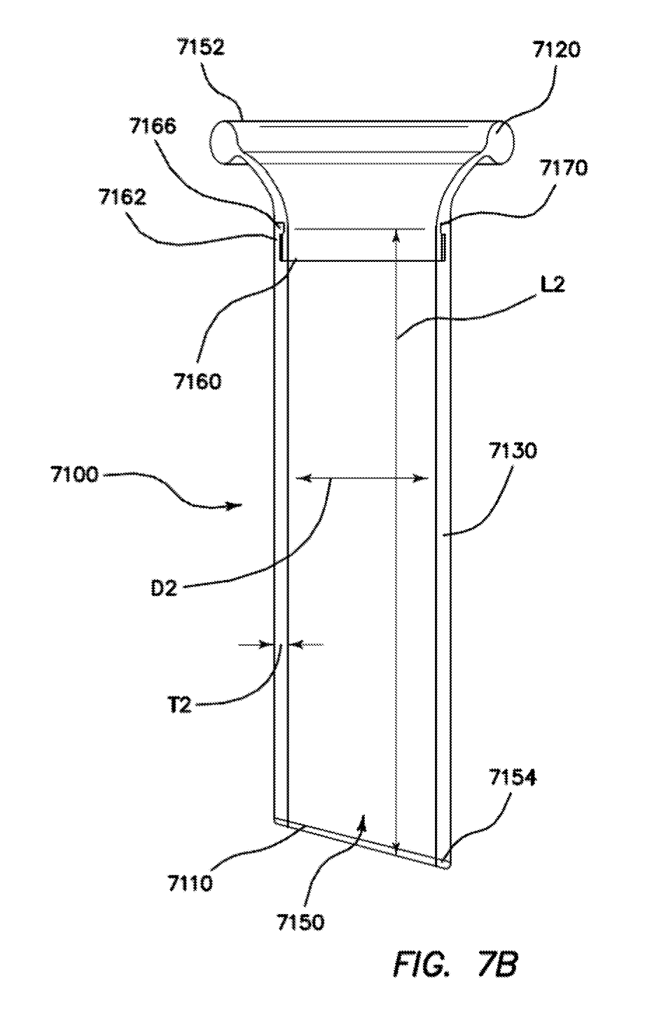

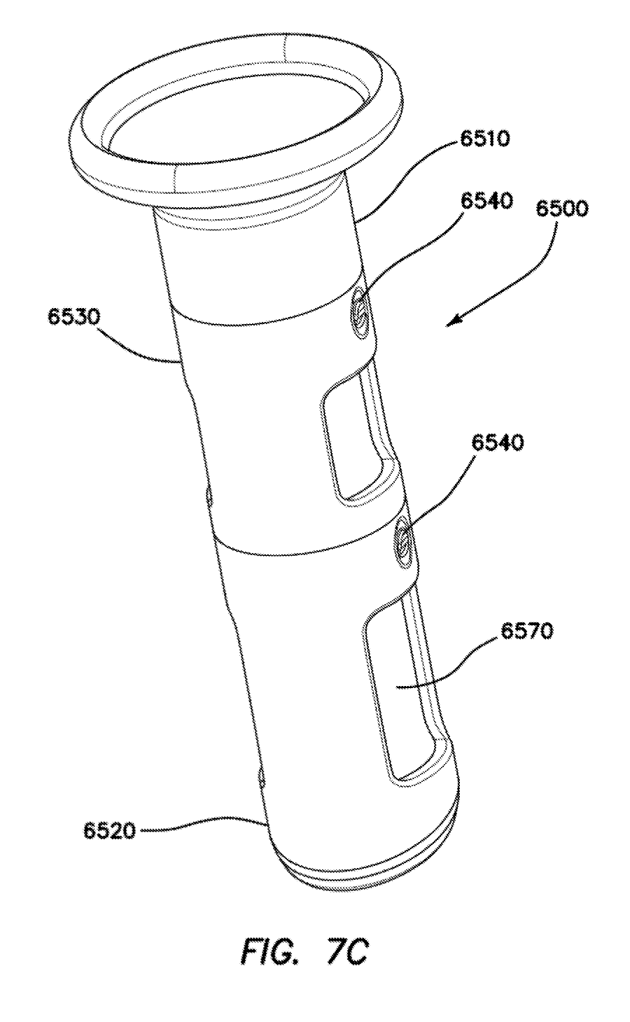

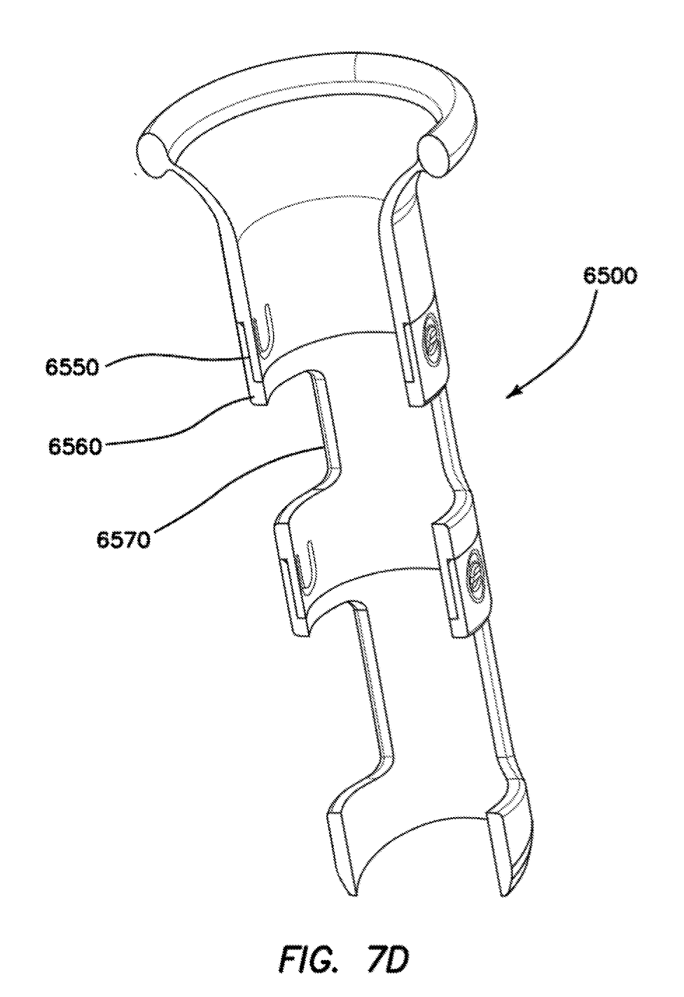

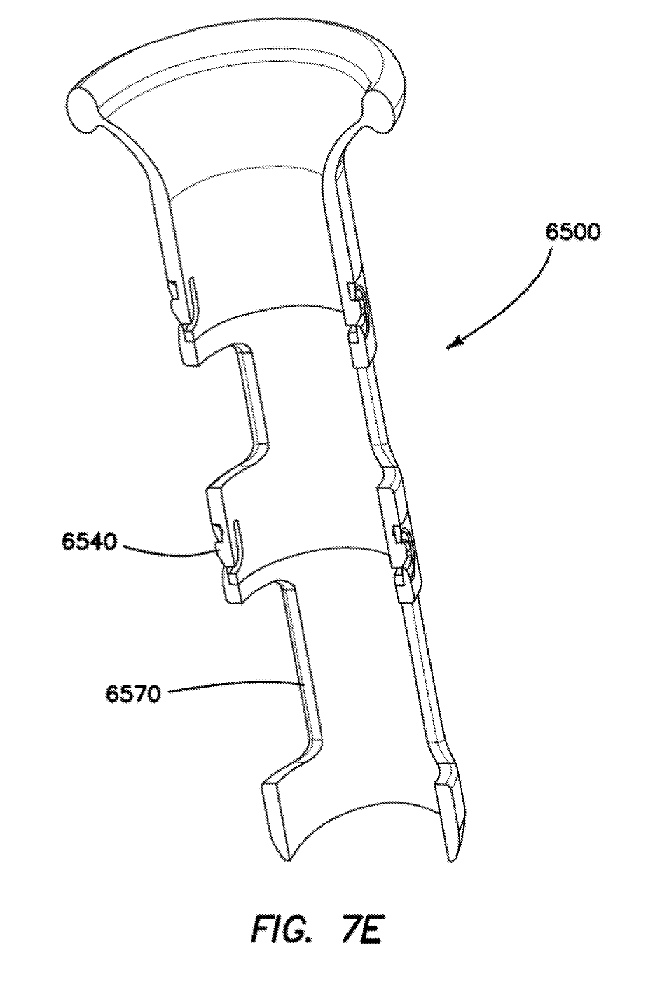

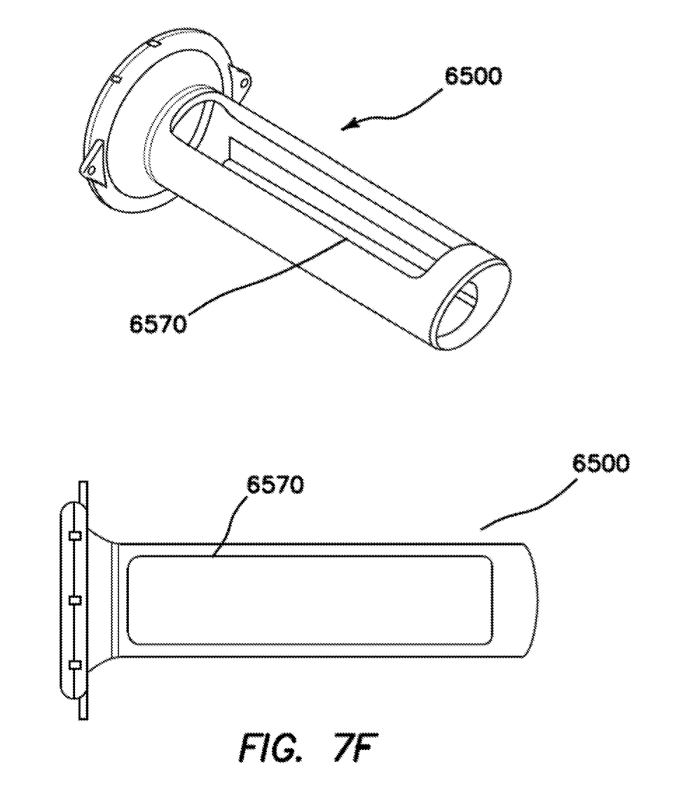

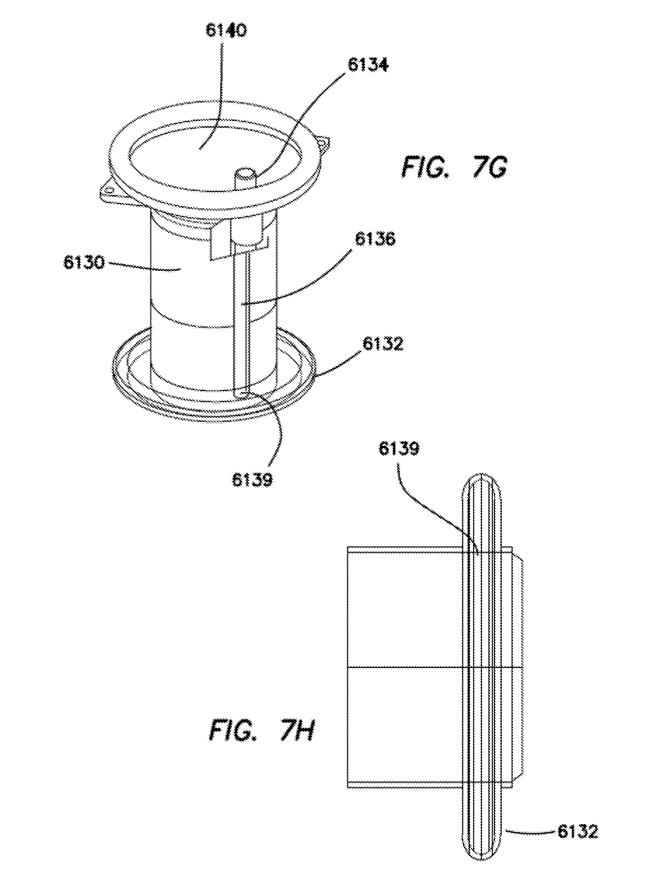

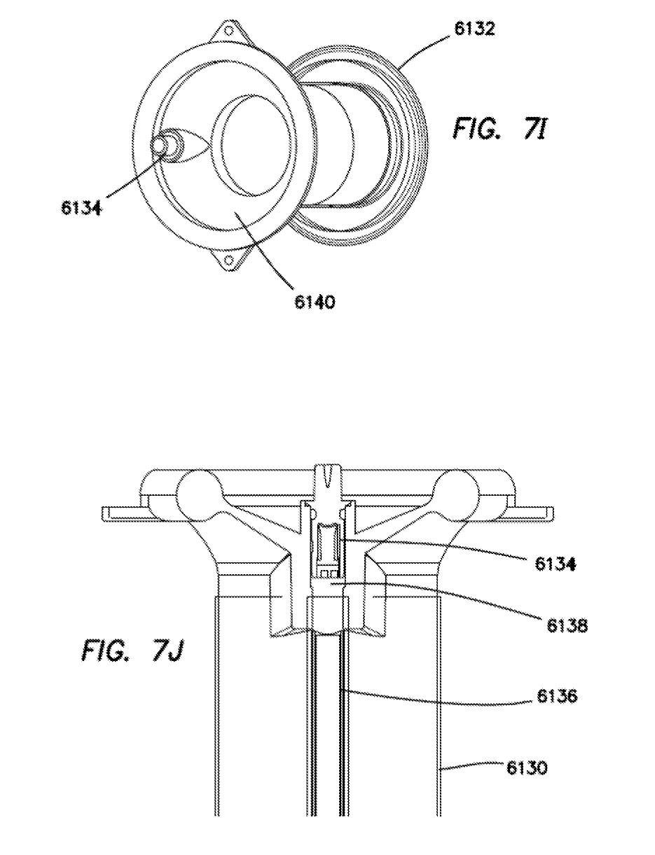

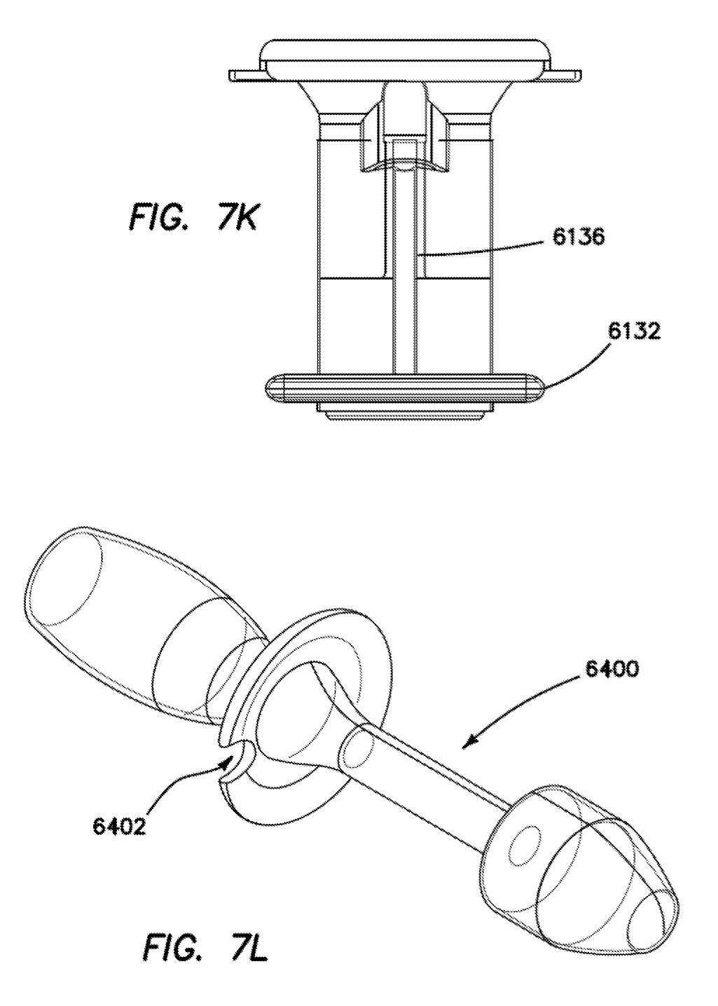

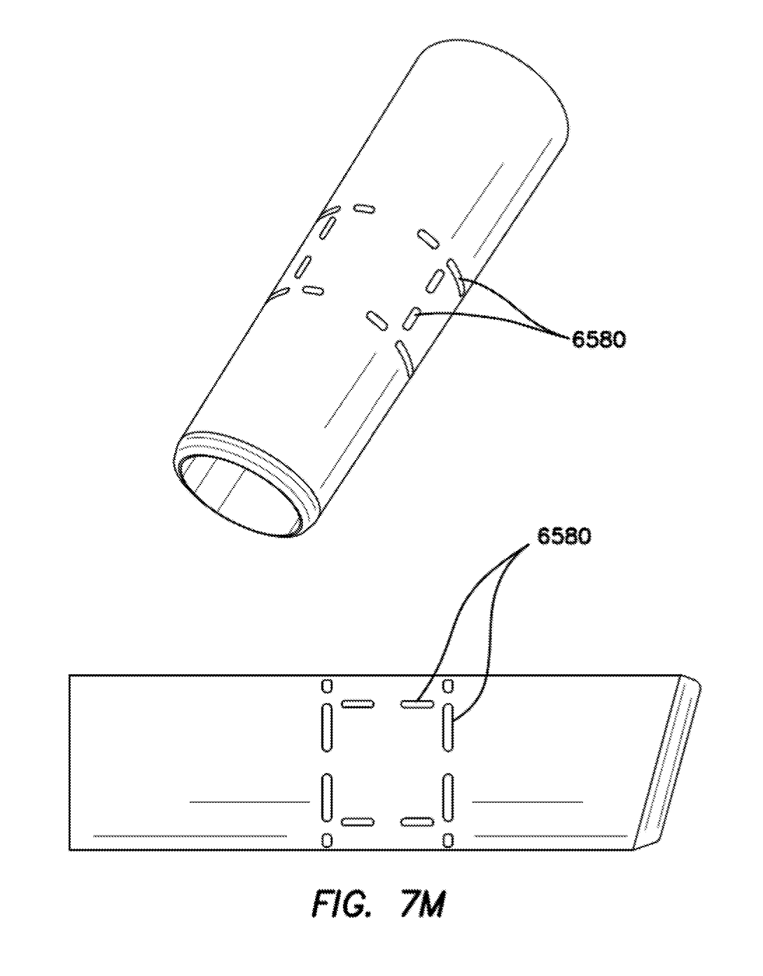

With reference to FIG. 7A, a cross-sectional view of a natural orifice access system including an access device 6100 and a removable cap 6200 is shown. In the illustrated embodiment, the tubular body 6130 is formed of a flexible material having a predetermined fixed length L, inner diameter D, and wall thickness T. The fixed length L, inner diameter D, and wall thickness T are selected to accommodate the anatomy of a natural orifice, such as the anal orifice of a majority of patients. It is contemplated that the access device 6100 can be scaled to different sizes for patients of different ages. Furthermore, in some embodiments, it is contemplated that the access device can include a telescopic tubular body such that the tubular body can be selectively positioned at a variety of lengths depending on patient anatomy and the location of the surgical site within the body cavity. Desirably, the wall thickness T and material of the tubular body 6130 are selected such that the tubular body 6130 is resilient enough to maintain the passage 6150 there through when positioned in the natural orifice. Moreover, desirably, the inner diameter, D is sufficiently large to accommodate multiple surgical instruments. For example, in embodiments of the access device 6100 adapted for use in a TEMS procedure, the inner diameter D and thickness T can be sized such that an outer diameter of the access device can be between approximately 30 mm and 70 mm, desirably between approximately 35 mm and 50 mm, and in one embodiment approximately 40 mm. Additionally, desirably, the fixed length L is sufficiently long such that the inner ring 6110 can be positioned at a surgical site within a body cavity and the outer ring 6120 can be positioned outside the natural orifice. In some embodiments, the fixed length L is of a length such that the device has an overall length between the proximal end 6152 and the distal end 6154 of between approximately 10 mm and approximately 100 mm, desirably between approximately 20 mm and 80 mm, more desirably between approximately 30 mm and 60 mm, and in one embodiment, approximately 40 mm.

With continued reference to FIG. 7A, in some embodiments, the annular groove 6122 can be open to an inner surface of the outer ring 6120. Thus, the access device 6100 can be formed of a flexible material in a single molding operation with the annular groove 6122 having an opening, and the reinforcing member 6124 can be subsequently inserted into the upper groove 6122.

With continued reference to FIG. 7A, in some embodiments, the access device 6100 can include a flexion region between the tubular body 6130 and the inner ring 6110, such as an undercut 6170. Advantageously, the flexion region can allow the inner ring 6110 to flex or rotate relative to the tubular body 6130 during insertion such that the inner ring 6110 presents a relatively small outer diameter in an insertion configuration and a relatively larger outer diameter in an undisturbed configuration.

In other embodiments, shown in FIG. 7G, the inner ring can comprise an inflatable member 6132 such as an annular balloon coupled to a gas or fluid source that can be selectively inflated and deflated between a deflated, relatively small diameter state for insertion and removal, and an inflated, relatively high diameter state for retention in a body cavity. An inflation port 6134, for example a check valve, affixed to the funnel portion 6140 of the access device, is connected to the inflatable member 6132 through a channel 6136 within the wall of the tubular body 6130. Fluid or gas introduced through the inflation port flows through the channel into the inflatable member to thereby inflate the member.

The channel 6136 runs through the tubular body, generally parallel to the longitudinal axis of the tubular body, with a proximal opening interacting with the inflation port 6134 and a distal opening 6139 into outer surface of the tubular body at the inflatable member. In one aspect, the inflation port 6134 may include a normally closed check valve having a spring-loaded plunger. In a further aspect, the check valve may include a Luer lock. It is contemplated that other inflation ports that are well known in the art may be used.

In this embodiment, the tubular body 6130 is preferably comprised of a relatively rigid material, such as a polycarbonate. The tubular body has an inflatable member at the distal end that may be created by heat shrinking polyolefin tubing around the outside of the tubular body. The distal end of the body/tubing assembly is then heated for approximately 30 to 40 seconds, and then placed inside a mold and injected with air to give the inflatable member an annular balloon shape as seen in FIG. 7H, or any other desired shape, depending on the configuration of the mold. The inflatable member 6132 should have sufficient impermeability properties to substantially prevent inflation gas or fluid from permeating through a wall of the inflatable member.

In one embodiment, the inflatable member 6132 may include a substantially toroid shape upon inflation. In another embodiment, the inflatable member may include a disc shape upon inflation. In another embodiment, the inflatable member 6132 may be a fluted balloon. Other shapes suitable for particular natural orifices will be appreciated by one skilled in the art.

In use, the inflatable member may be inflated after the access device is disposed within the natural orifice by inserting a syringe into the valve 6134 located at the proximal end 6138 of the channel within the tubular body (see FIG. 7I). As shown in FIGS. 7J and 7K, the port leads into the channel 6136, which allows the fluid or gas from the syringe to travel to the inflatable member 6132. In this embodiment, the optional obturator 6400 may be modified with an indent 6139 to provide clearance for the inflation port, as shown in FIG. 7L.

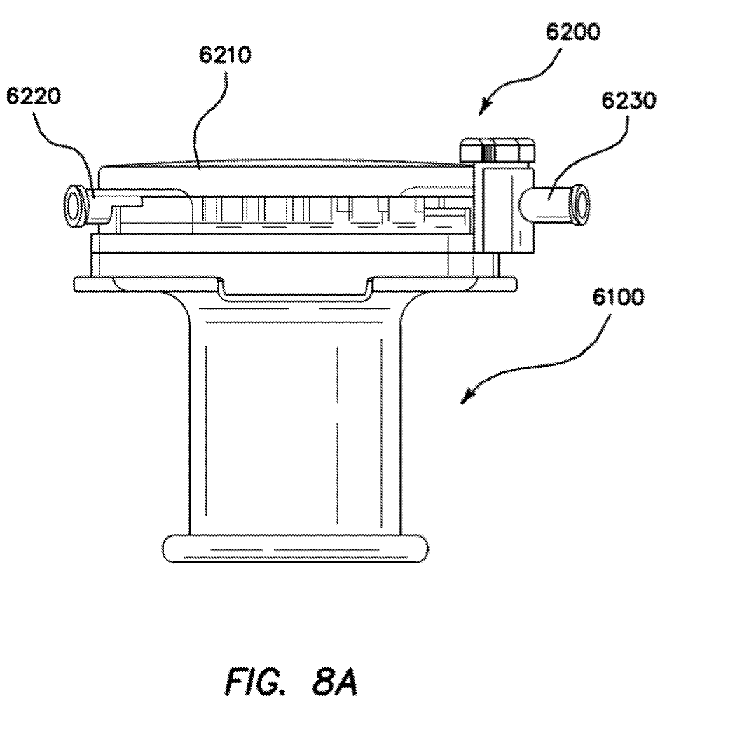

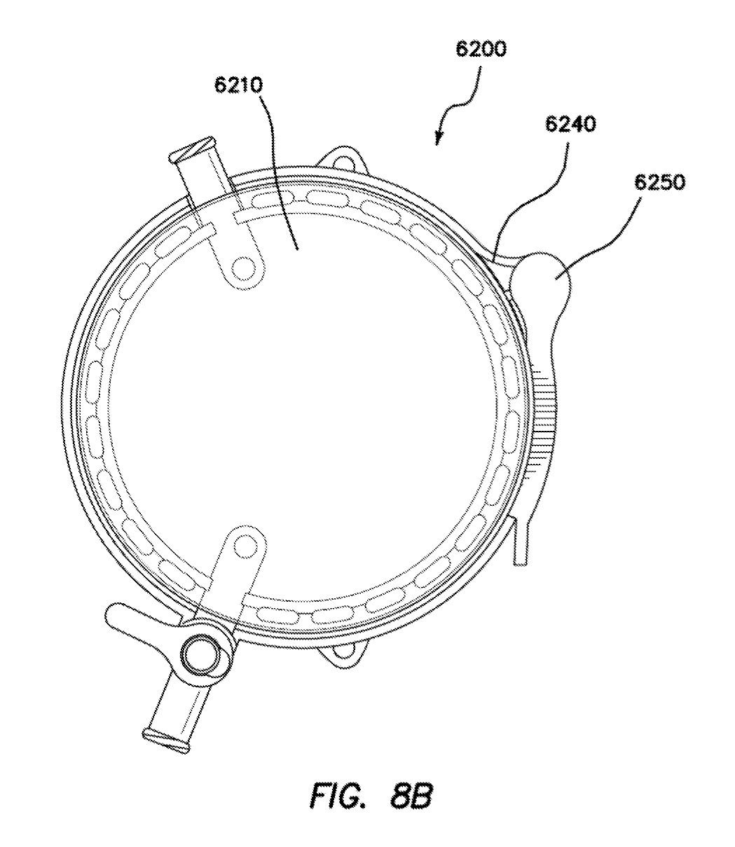

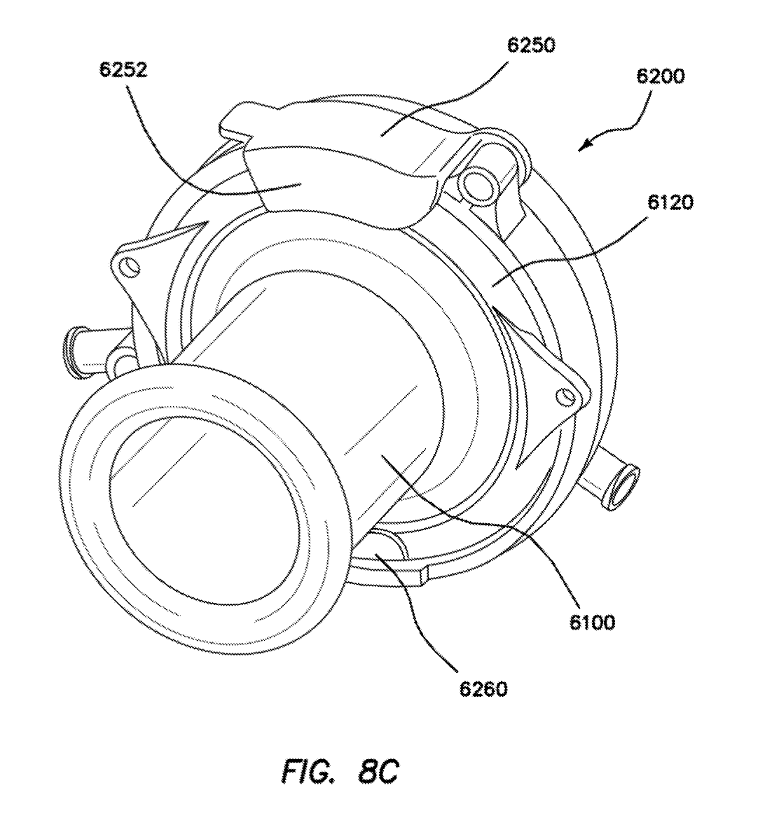

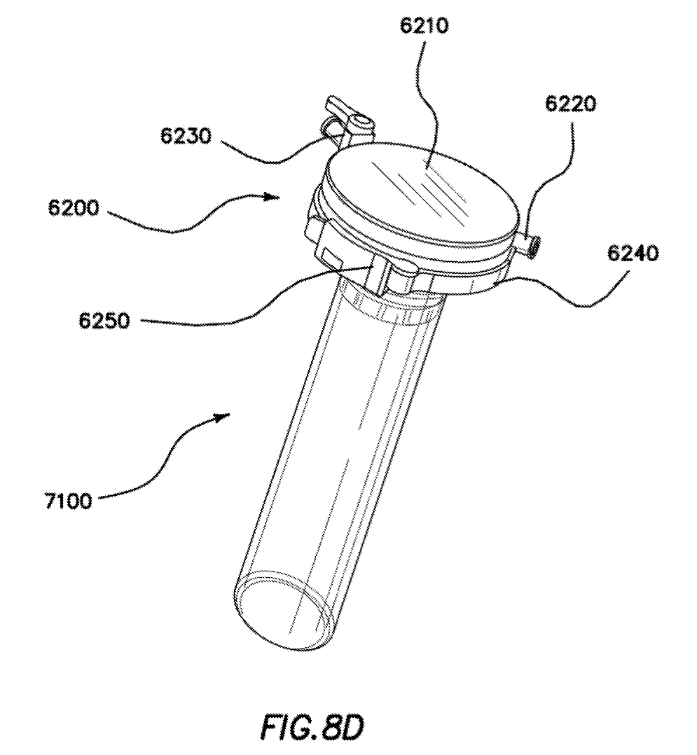

With reference to FIG. 8A, a side view of a natural orifice access device having a cap 6200 removably coupled to an access device 6100 is illustrated. In the illustrated embodiment, the cap 6200 comprises a sealable access surface 6210 such as a gel pad surface as described in further detail herein. In certain embodiments, the cap 6200 can also comprise at least one gas or fluid port 6220, 6230. In the illustrated embodiment, the cap 6200 comprises two gas or fluid ports 6220, 6230, such that one port can be used for gas insufflation and the other port can be used for ventilation for example when electrosurgery is performed through the access device. In certain embodiments, at least one of the gas or fluid ports 6220, 6230 comprises a valve such as a stopcock valve to selectively control the flow of fluid there through.

With reference to FIG. 8B, a top view of the natural orifice access system is illustrated. The sealable access surface 6210 can be encircled by and restrained by an annular frame 6240 such as a split ring having a clamp 6250. The clamp 6250 can be movable between an open configuration in which the cap 6200 is selectively removable from the access device 6100 and a clamped configuration in which the cap 6200 can be secured to the access device 6100. For example, the annular frame 6240 can be positioned peripherally around the outer ring 6120 with the clamp 6250 in the open configuration and the clamp moved to the clamped configuration to sealingly fix the cap 6200 to the access device 6100. Accordingly, the cap 6200 can be easily removed during a surgical procedure to facilitate removal of excised tissue from a surgical site through the access device 6100.

With reference to FIG. 8C, a perspective view of the natural orifice access system is illustrated. In the illustrated embodiment, the clamp 6250 can have a distal flange 6252 positioned to interface with the outer ring 6120 of the access device when the clamp is in the clamped configuration. As illustrated, the clamp 6250 engages a distal surface of the outer ring 6120 of the access device 6100. In some embodiments, the annular frame 6240 can further comprise at least one distal flange sized and positioned to interface with an access device. In the illustrated embodiment, the annular frame 6240 comprises a distal flange 6260 positioned to engage a distal surface of the outer ring 6120 of the access device. As illustrated, the flange 6260 is generally diametrically opposed to the distal flange of the clamp 6250. In other embodiments, the annular frame 6240 can include more than one distal flange positioned substantially equally spaced about the periphery of the annular frame 6240 or spaced irregularly about the periphery of the annular frame.

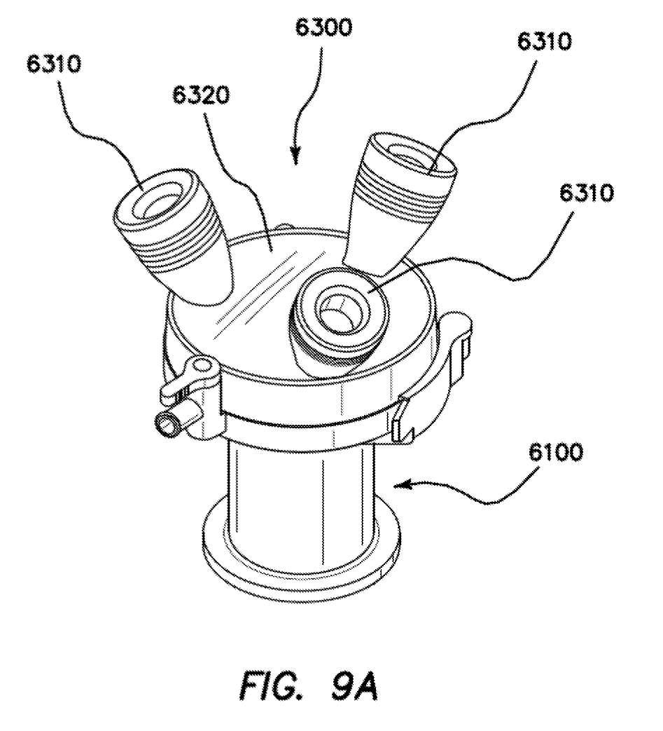

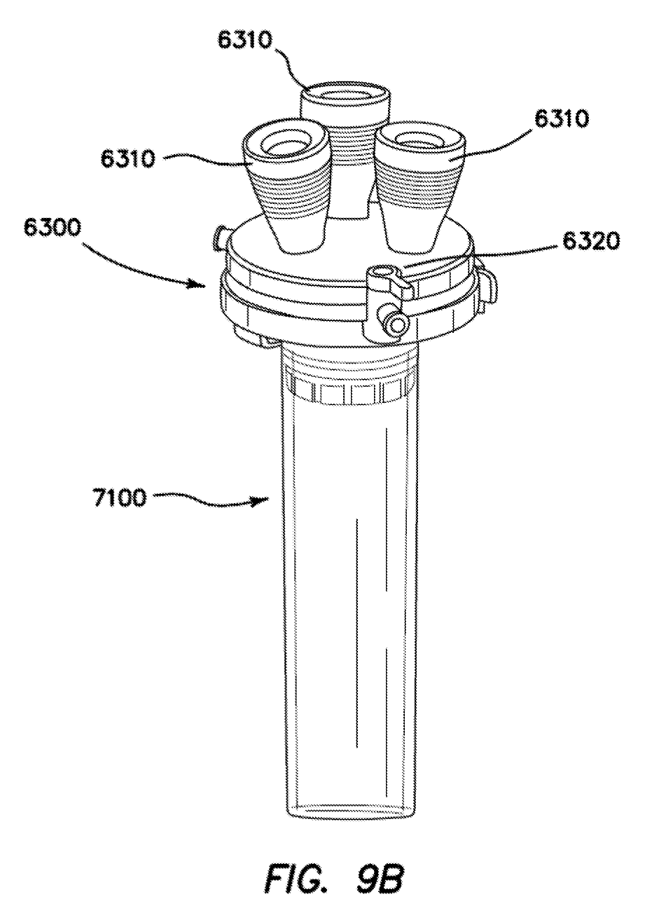

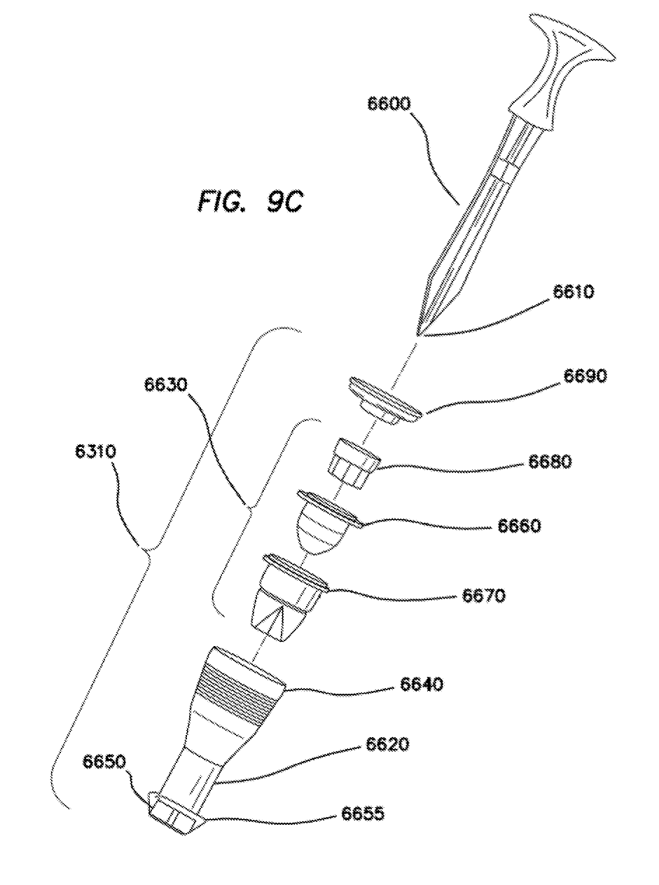

With reference to FIG. 9A, another embodiment of natural orifice access system is illustrated with a cap 6300 removably coupled to an access device 6100 such as that described above with respect to FIGS. 6A-6C, 7A, 8A-8C, and 9A. In the illustrated embodiment, the cap 6300 includes multiple trocar access devices 6310 positioned through an access surface 6320 thereof. Advantageously, the multiple trocar access devices 6310 allow for easy placement and manipulation of multiple laparoscopic instruments in a surgical site through a single natural orifice.

In some embodiments, the inner ring 6110 and the outer ring 6120 independently have different footprint shapes and/or footprint diameters. For example, in the embodiment illustrated in the embodiment of access device 7100 illustrated in FIGS. 6D-F, 7B, 8D, and 9B, the inner ring 7110 can be substantially flush with the tubular body 7130 while the outer ring 7120 can be an annular member having a generally circular cross-section. An inner ring 6110 with a larger diameter permits a greater retraction force, but is more difficult to insert and remove from a body cavity.

With reference to FIGS. 6D-6F, in some embodiments, a natural orifice access device 7100 can be adapted for use in a transanal endoscopic microsurgery (TEMS) procedure. The access device 7100 comprises an inner or distal ring 7110, an outer or proximal ring 7120, a tubular body 7130, and a funnel segment 7140 extending between and coupling the inner ring 7110 and the outer ring 7120. The tubular body 7130 comprises a relatively flexible material such as a KRATON® polymer or a silicone rubber material, which is substantially cylindrical in the illustrated embodiment. In other embodiments, the tubular body 7130 has another shape, for example, an oval cross section. Some embodiments of the tubular body 7130 comprise one or more coatings that provide additional functionality, for example, an anti-microbial coating.

In the illustrated embodiment, the inner ring 7110 is substantially flush with a distal end of the tubular body 7130 such that the access device 7100 has a generally tubular configuration extending distally of the funnel segment 7140 to the distal end. Embodiments of the inner ring 7110 are sufficiently flexible and compliant to be compressed and/or deformed for insertion into a body orifice such as a patient's anus during a transanal surgical procedure. When subsequently released within an associated body cavity, the inner ring 7110 substantially returns to its original shape or footprint. In some embodiments, the inner ring 7110 assumes a substantially circular shape substantially flush with the generally cylindrical tubular body 7130 in a relaxed state, for example, when released within a body cavity. In other embodiments, the inner ring 7110 has another shape in the relaxed state, for example, an oval. The inner ring 7110 assumes a different shape when compressed for insertion through an incision or body orifice, for example, a substantially oval shape, a generally linear shape, a tear-drop shape, or another suitable shape. In other embodiments, the inner ring 7110 is substantially rigid, that is, non-compliant under the ordinary conditions under which it is used.

With continued reference to FIGS. 6D-6F, in some embodiments, the inner ring 7110 can be shaped and configured to facilitate insertion through a natural orifice. For example, in the illustrated embodiment, the inner ring 7110 can include a radiused edge to facilitate atraumatic entry through a natural orifice. In other embodiments, the inner ring 7110 can include a beveled edge to facilitate entry through a natural orifice. Furthermore, in the illustrated embodiment, the inner ring 7110 can be formed at an angle transverse to a longitudinal axis defined by the tubular body 7130. Advantageously, such an angled inner ring 7110 can facilitate insertion of the access device 7100 through a natural orifice. In other embodiments, the inner ring 7110 can be substantially perpendicular to the longitudinal axis defined by the tubular body.

With continued reference to FIGS. 6D-6F, the outer ring 7120 is proximal the funnel section 7140. In the illustrated embodiment, the outer ring 7120 has a substantially circular footprint. As further discussed herein, the outer ring 7120 can be sized and configured to sealingly couple to a cap or other access device thereon. In some embodiments, as discussed above with reference to the embodiments of FIGS. 6A-6C, one or more suture points can be disposed on the access device 7100 adjacent the outer ring 7120.

With continued reference to FIGS. 6D-6F, the tubular body 7130 can have a generally circular profile defining a generally cylindrical passage 7150. The generally cylindrical passage 7150 is desirably large enough to accommodate more than one laparoscopic instrument there through such that a single natural orifice access device can be used to provide access for multiple surgical instruments in a body cavity. Moreover, generally cylindrical passage 7150 is desirably large enough such that multiple surgical instruments positioned there through can be translated or pivoted relative to one another, allowing a surgeon to manipulate the instruments as desired during a surgical procedure. The generally cylindrical passage extends between a proximal end 7152 of the access device 7100 adjacent the outer ring 7120 to a distal end 7154 of the access device 7100 adjacent the inner ring 7110 (FIG. 6D).

With reference to FIG. 6D, in the illustrated embodiment, the funnel segment 7140 provides a diametric reduction between the relatively large diameter of the outer ring 7120, which is sized and configured to be removably coupled to an access device such as a cap, and the relatively smaller diameter of the passage 7150, which is sized to fit within a natural orifice with minimal distention of the orifice. The funnel segment 7140 has an inner surface 7142 which can provide a bearing surface for an obturator used to advance to the access device 7100 into a body cavity. In some embodiments, the funnel segment 7140 can have a substantially linear taper between the relatively large diameter and the relatively smaller diameter such that the inner surface 7142 is a frusto-conical segment. In other embodiments, the funnel segment 7140 can have a curved profile between the relatively large diameter and the relatively smaller diameter.

In some embodiments, a natural orifice access system can include an access device 7100 and an optional obturator, such as described above with reference to FIG. 6G. The obturator can have a proximal bearing surface 6410 sized and configured to bear against the inner surface 7142 of the funnel segment 7140 and a distal dilation surface 6420 sized and configured to expand a natural orifice for passage of the access device 7100. Thus, during insertion of the access device 7100 into a natural orifice, the dilation surface expands a pathway to a surgical site in a body cavity while the obturator bears on the inner surface 7142 of the funnel segment 7140 to advance the access device 7100 into position in the surgical site. Furthermore, in some embodiments, the obturator can have a handle 6430 at a proximal end thereof adapted to facilitate selective twisting or rotation of the obturator about a longitudinal axis thereof during insertion.

In an alternative embodiment, shown in FIG. 6I, the obturator 6405 includes a straight shaft piece 6425 between the distal dilation surface 6420 and the proximal bearing surface 6410 that facilitates dilation of the natural orifice prior to inserting the access device. It can then be combined with the access device 7100 to help ease insertion, as shown in FIG. 6J.

With reference to FIG. 7B, it can be desirable that the outer ring 7120 is relatively stiff compared with the relatively flexible tubular body 7130 of the access device 7100 so that the outer ring 7120 can sealingly engage an access device such as a cap. In the illustrated embodiment, the access device 7100 is formed in a multiple-shot molding process. For example, in the illustrated embodiment, an inner segment of the access device 7100 defined by the tubular body 7130 and the inner ring 7110 is formed in one molding operation from a flexible material, and an outer segment of the access device 7100 defined by the funnel segment 7140 and the outer ring 7120 is formed in another molding operation from a relatively rigid material such as a polycarbonate material or other suitable material.

In other embodiments, a multiple-shot molding process can be varied such that the resulting inner and outer segments are different from those of the illustrated embodiment. For example, in certain embodiments, the inner segment can include the tubular body 7130, the inner ring 7110, and a portion of the funnel segment 7140, while the outer segment can include a portion of the funnel segment 7140 and the outer ring 7120. In certain other embodiments, the inner segment can include the inner ring 7110 and a portion of the tubular body 7130, while the outer segment can include a portion of the tubular body 7130, the funnel segment 7140, and the outer ring 7120.

With reference to FIGS. 6D and 7B, an access device 7100 formed in a multiple-shot molding process can include one or more retention members 7160 on the inner segment and the outer segment to maintain the position of the inner segment relative to the outer segment. For example, in some embodiments, a distal end of the outer segment can include one or more protrusions 7162 extending radially outwardly from the funnel segment 7140 and one or more recesses 7164 recessed radially inwardly from the funnel segment 7140 at an interface region of the inner segment and the outer segment of the access device 7100. In the illustrated embodiment, the distal end of the outer segment includes a plurality of protrusions 7162 alternating with a plurality of recesses 7164 there between. Moreover, in some embodiments, the outer segment can include an annular groove 7170 formed in the funnel segment 7140 at an interface region of the inner segment and the outer segment of the access device 7100. The inner segment of the access device 7100 can include an annular member 7166 disposed within and matingly engaging the groove 7170 to maintain the position of the inner segment relative to the outer segment.

With reference to FIG. 7B, a cross-sectional view of access device 7100 is shown. In the illustrated embodiment, the tubular body 7130 is formed of a flexible material having a predetermined fixed length L2, inner diameter D2, and wall thickness T2. The fixed length L2, inner diameter D2, and wall thickness T2 are selected to accommodate the anatomy of a natural orifice, such as the anal orifice of a majority of patients. It is contemplated that the access device 7100 can be scaled to different sizes for patients of different ages. Furthermore, in some embodiments, it is contemplated that the access device can include a telescopic tubular body such that the tubular body can be selectively positioned at a variety of lengths depending on patient anatomy and the location of the surgical site within the body cavity. Desirably, the wall thickness T2 and material of the tubular body 7130 are selected such that the tubular body 7130 is resilient enough to maintain the passage 7150 there through when positioned in the natural orifice. Moreover, desirably, the inner diameter, D2 is sufficiently large to accommodate multiple surgical instruments. For example, in embodiments of the access device 7100 adapted for use in a transanal surgical procedure, the inner diameter D2 and thickness T2 can be sized such that an outer diameter of the access device can be between approximately 30 mm and 70 mm, desirably between approximately 35 mm and 50 mm, and in one embodiment approximately 40 mm.

Additionally, desirably, the fixed length L2 is sufficiently long such that the inner ring 7110 can be positioned at a surgical site within a body cavity and the outer ring 7120 can be positioned outside the natural orifice. In some embodiments, the fixed length L2 is of a length such that the device has an overall length between the proximal end 7152 and the distal end 7154 of between approximately 100 mm and approximately 200 mm, desirably between approximately 120 mm and 180 mm, more desirably between approximately 140 mm and 160 mm, and in one embodiment, approximately 150 mm.

With reference to FIG. 8D, a perspective view of a natural orifice access system having a cap 6200 substantially similar to that described with respect to FIGS. 8A-8C removably coupled to an access device 7100 is illustrated. In the illustrated embodiment, the cap 6200 comprises a sealable access surface 6210 such as a gel pad surface as described in further detail herein. In certain embodiments, the cap 6200 can also comprise at least one gas or fluid port 6220, 6230. In the illustrated embodiment, the cap 6200 comprises two gas or fluid ports 6220, 6230, such that one port can be used for gas insufflation and the other port can be used for ventilation for example when electrosurgery is performed through the access device. In certain embodiments, at least one of the gas or fluid ports 6220, 6230 comprises a valve such as a stopcock valve to selectively control the flow of fluid there through.

With continued reference to FIG. 8D, a top view of the natural orifice access device is illustrated. The sealable access surface 6210 can be encircled by and restrained by an annular frame 6240 such as a split ring having a clamp 6250. The clamp 6250 can be movable between an open configuration in which the cap 6200 is selectively removable from the access device 7100 and a clamped configuration in which the cap 6200 can be secured to the access device 7100. For example, the annular frame 6240 can be positioned peripherally around the outer ring 7120 with the clamp 6250 in the open configuration and the clamp moved to the clamped configuration to sealingly fix the cap 6200 to the access device 7100. Accordingly, the cap 6200 can be easily removed during a surgical procedure to facilitate removal of excised tissue from a surgical site through the access device 7100.

With reference to FIG. 9B, another embodiment of natural orifice access device is illustrated can include a cap 6300 substantially similar to that described above with reference to FIG. 9A removably coupled to an access device 7100 such as that described above with respect to FIGS. 6D-F, 7B, and 8D. The cap 6300 can include multiple trocar access devices 6310 positioned through an access surface 6320 thereof. Advantageously, the multiple trocar access devices 6310 allow for easy placement and manipulation of multiple laparoscopic instruments in a surgical site through a single natural orifice.

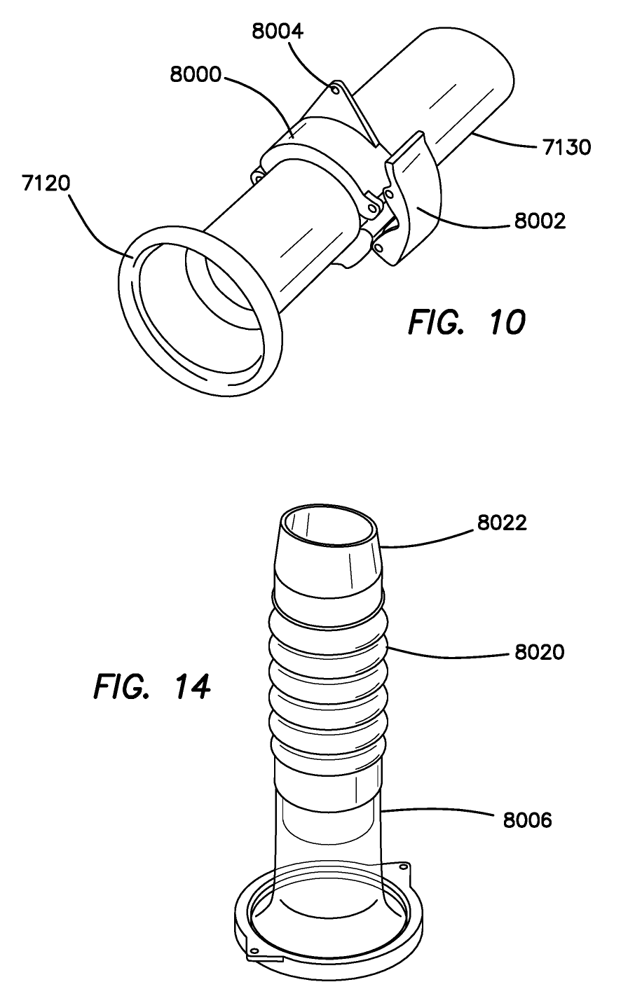

As discussed herein, the access devices shown in FIGS. 7A and 7B can include a telescopic tubular body such that the tubular body can be selectively positioned at a variety of lengths depending on patient anatomy and the location of the surgical site within the body cavity. Alternatively, the access device may have a fixed-length tubular body, but may be adapted to slide through a separate clamping device, shown in FIG. 10. In this embodiment, a clamping device 8000 having suturing tabs 8004 is sutured to the patient around the natural orifice. The tubular body of the access device is inserted into the natural orifice through the clamping device and moved distally into the orifice until the distal end is positioned near the surgical site. The access device is then secured within the clamping device by moving a latch 8002 on the clamping device from an open to a closed position, to prevent further movement of the access device.

In another embodiment, illustrated in FIG. 7C, the tubular body may be formed in sections of varying length that slidingly engage and snap lock together to provide a variety of lengths, depending of the number and size of the sections selected and assembled. With reference to FIG. 7C, a perspective view of an access device 6500 is shown having three sections: an outer ring section 6510, an inner ring section 6520, and an intermediate section 6530 disposed between the other two sections. The three sections are held together by a snap lock mechanism 6540. Each section terminates at the distal end with an annular groove 6550 that slidingly engages with the proximal end 6560 of the next section, best shown in the cross section view of FIG. 7D. The snap lock mechanism is shown in cross-section in FIG. 7E. The tubular body of the embodiment shown in FIG. 7C-E is preferably formed from a relatively stiff material, such as a polycarbonate.

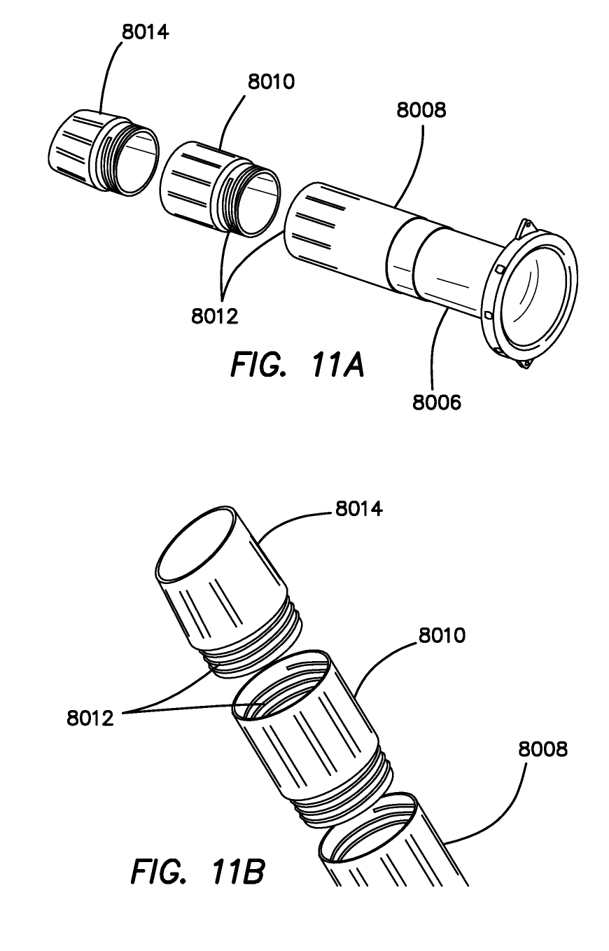

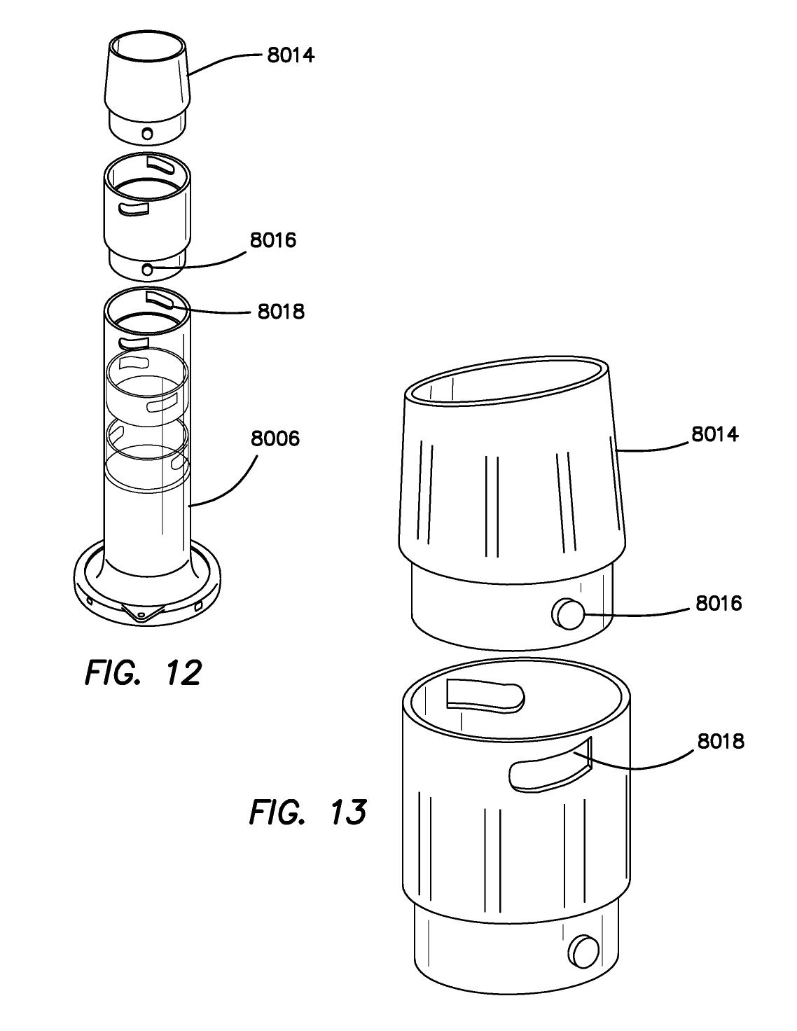

In other embodiments, the length of the tubular body may be adjusted by adding or removing sections that attach to each other using threading (FIG. 11) or a twist-lock mechanism (FIGS. 12 and 13). In FIG. 11, a base 8006 comprising an outer ring, funnel segment and a portion of the tubular body terminates in a threaded portion 8008, preferably comprising a polycarbonate. One or more shorter threaded sections 8010 may be added to the distal end of the base to yield the desired length of the tubular body. The threaded portion of the base 8008 and the threaded sections 8008 include screw threads 8012 that permit the base and section(s) to be screwed together. Optionally, a tapered tip section 8014, preferably comprising a polycarbonate, can be attached at the distal end of the tubular body to facilitate placement of the access device in the patient.

In FIG. 12-13, the tubular body of the base 8006 may be extended by adding sections through a twist-lock mechanism, facilitating rapid addition or removal of sections. A tapered tip section 8014 may also be added. The twist lock mechanism comprised a detent 8016 and a slot 8018 into which the detent may be inserted. Twisting the two pieces slides the detent into a locked position in the slot to secure the pieces together.

Another embodiment of an adjustable length access device is shown in FIG. 14. In this embodiment, the tubular body 8020 of the access device comprises collapsible tubing. By either compressing or pulling the tubular body axially, a surgeon is able to adjust the channel length to reach the exact lesion depth. The collapsible tubing section can be blow-molded from polyethylene. Optionally, a tapered tip 8022, preferably formed from polycarbonate, may be attached to the distal end of the collapsible tubular body to assist in introduction into the patient. The collapsible tubing can be adjusted forward and backward several times throughout the procedure in order to reach different lesions.

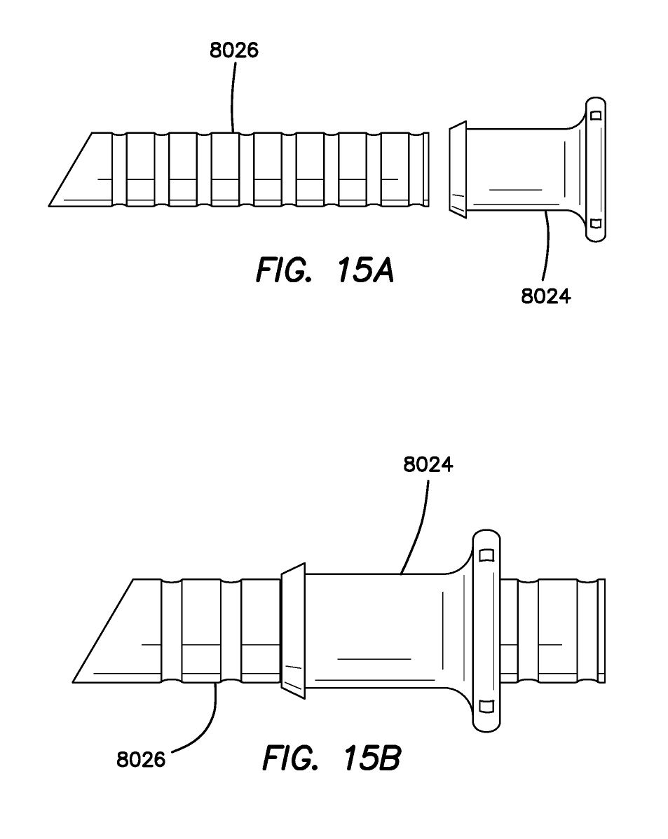

In the embodiment shown in FIG. 15, an access device 8024 similar to that described in FIG. 6A may be extended in length by sliding a separate segmented channel 8026 though the access device. The segmented channel has an outer diameter adapted to fit within the inner diameter of the access device so that it may slide forward and back when disposed within the access device, but with a fit sufficiently snug so as to avoid unintended displacement. Once the segmented channel is manipulated through the access device to the desired location, excess channel material extending through the outer ring of the access device may be removed with a scalpel or other cutting device.

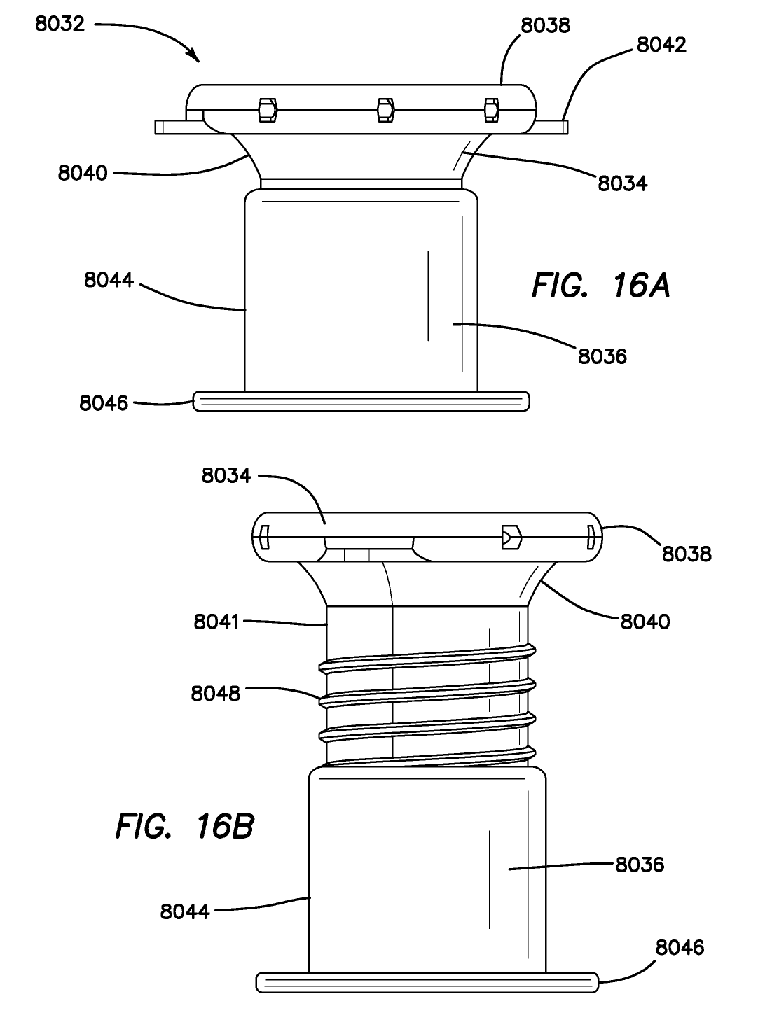

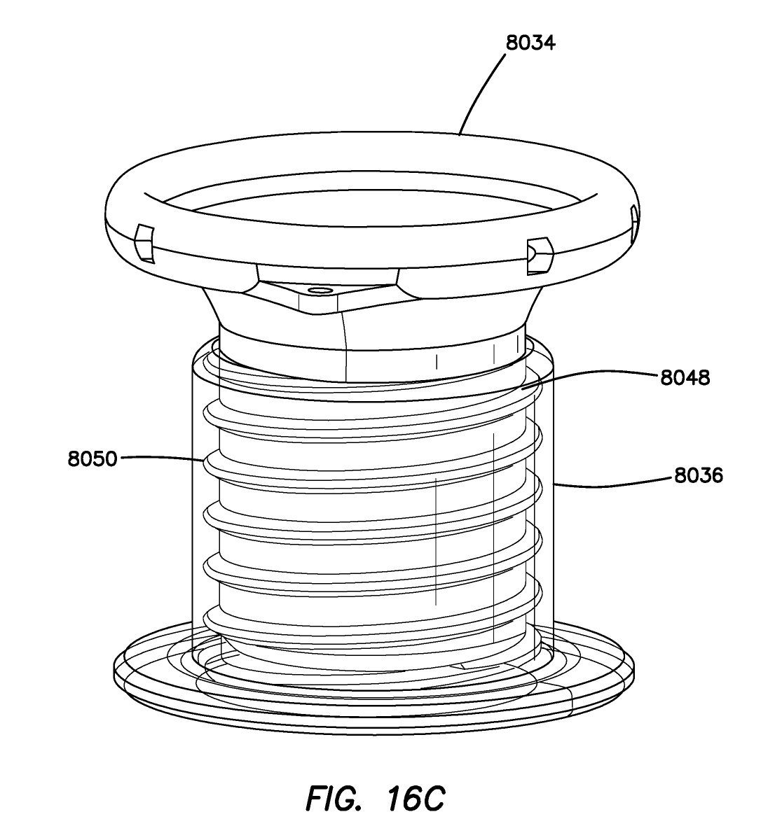

In some embodiments of the present invention, the access device comprises two slidably engageable portions. One such embodiment is shown in FIG. 16A. In this embodiment, the access device 8032 comprises an exterior portion 8034 disposed within an interior anchoring portion 8036. The exterior potion 8034 comprises an outer ring 8038, which may engage with a cap portion (not shown), a funnel portion 8040, a tubular channel 8041 disposable within the interior portion 8036 (not shown in FIG. 16A, but see FIG. 16B) and, optionally, suture points 8042. The interior portion 8036 comprises a tubular body 8044 and an inner ring 8046, which facilitates anchoring the access device in the natural orifice.

As can be best seen in FIGS. 16B and 16C, the exterior portion of this embodiment includes external (male) threads 8048 disposed around the outer surface of the tubular channel 8041, which engage with internal (female) threads 8050 disposed around the inner surface of the tubular body 8044 of the interior portion 8036. To adjust the access device length, a user can rotate the exterior portion relative to the interior portion. In the embodiment shown, rotating the external threads clockwise will shorten the device; rotating the external threads counterclockwise will lengthen the device.

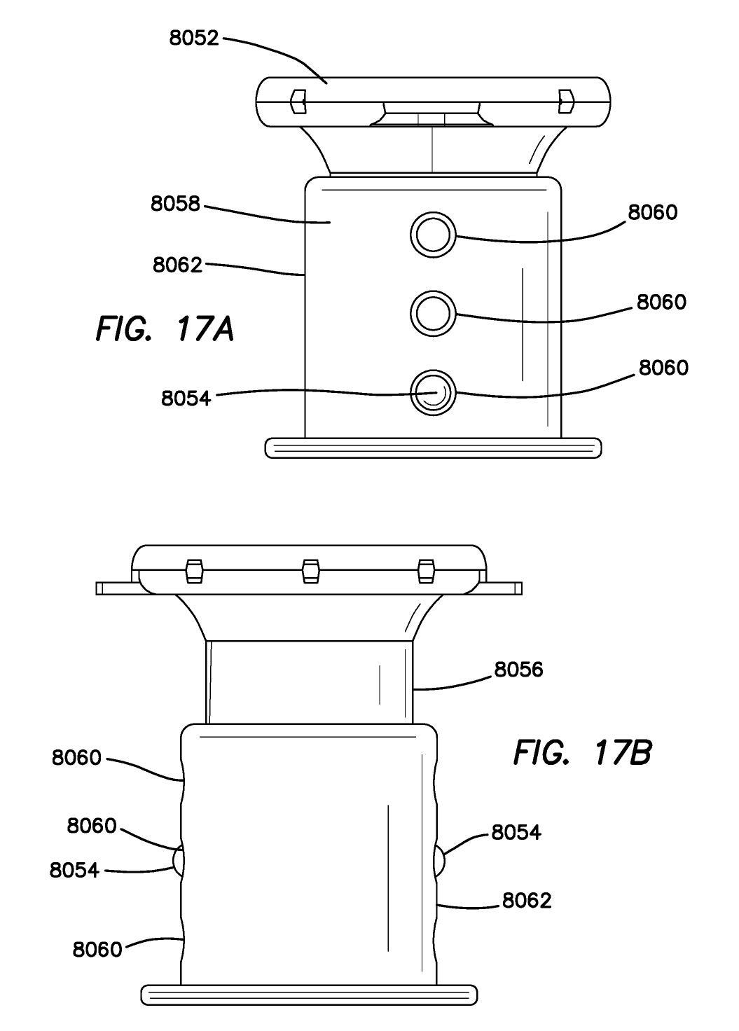

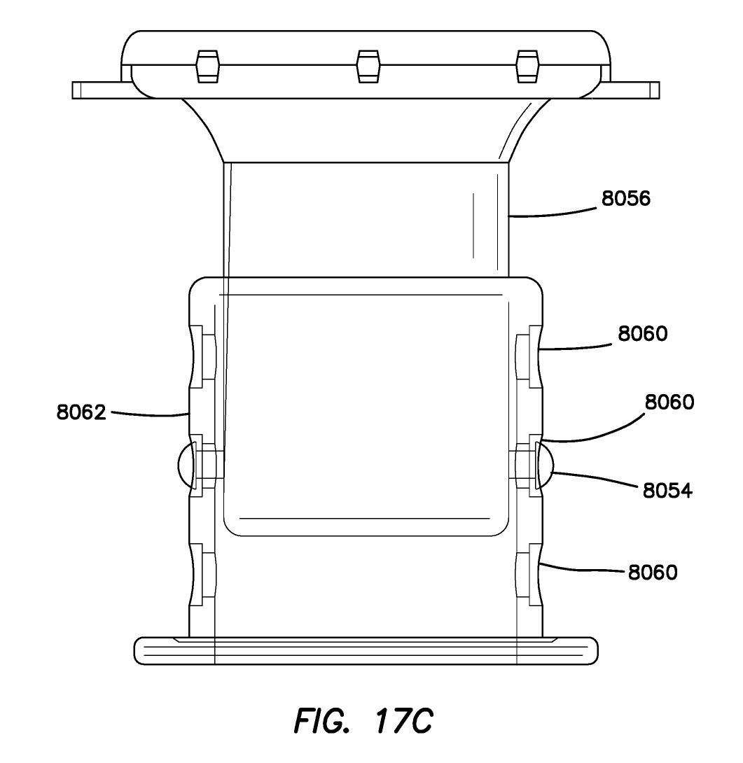

Another embodiment of the two-piece access device is shown in FIGS. 17A-C. In this embodiment, the exterior portion 8052 includes at least one pin 8054 on the outer surface of the tubular channel 8056. The interior portion 8058 includes two or more holes 8060 in the tubular body 8062 adapted to receive the pin(s) 8054 in a snap-fit. In the embodiment shown (best seen in FIGS. 17B and C), two pins 8054 are disposed on opposite sides of the tubular channel while two sets of three holes are disposed through opposite sides of the tubular body. To adjust the length of the access device, the user inserts the exterior portion 8052 into the interior portion 8058, slides the two portions relative to each other to achieve the desired length, then rotates the two portions relative to each other to guide the pin(s) into the hole(s) to secure the two portions.

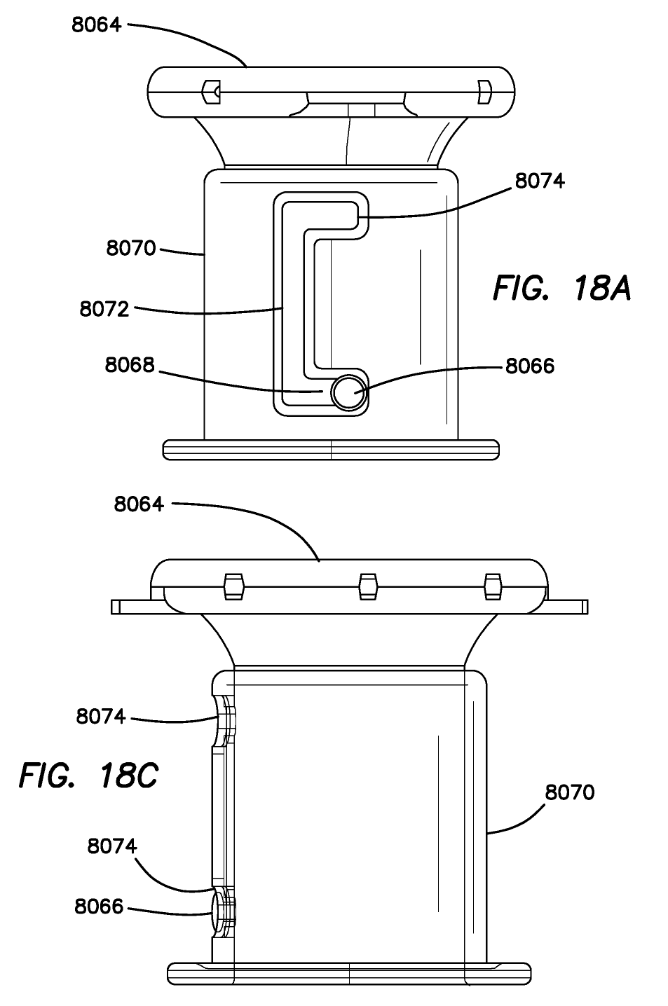

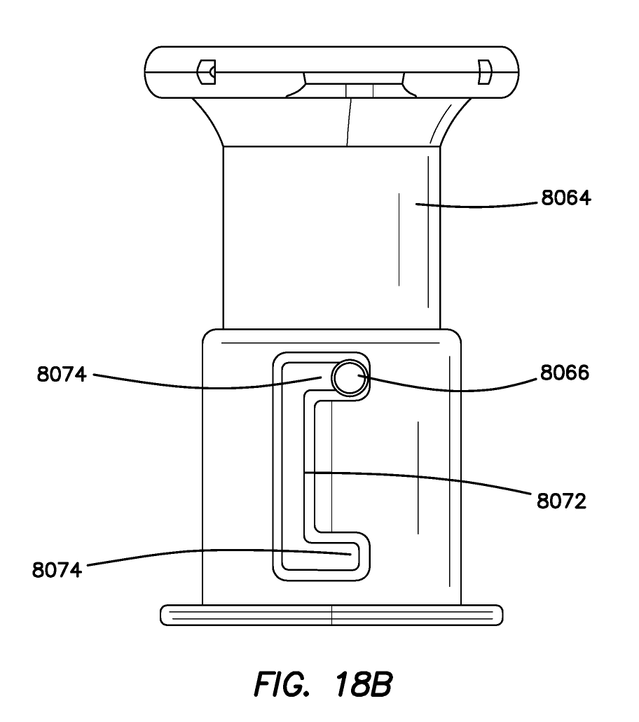

Still another embodiment of the two-piece access device is shown in FIGS. 18A-C. In this embodiment, the access device length is incrementally adjustable using a pin and groove combination. The exterior portion 8064 includes as least one pin 8066 on the outer surface of the tubular channel 8068. The interior portion 8070 includes a groove 8072 having at least two recess slots 8074, adapted to receive the pin 8066. To adjust the length of the access device, a user rotates the exterior portion 8064 to position the pin 8066 in the groove 8072 and slides the portions relative to each other until the desired length is achieved. The external portion is then rotated in the opposite direction to secure the pin in the closest recess 8074 to lock the two portions and prevent further sliding.

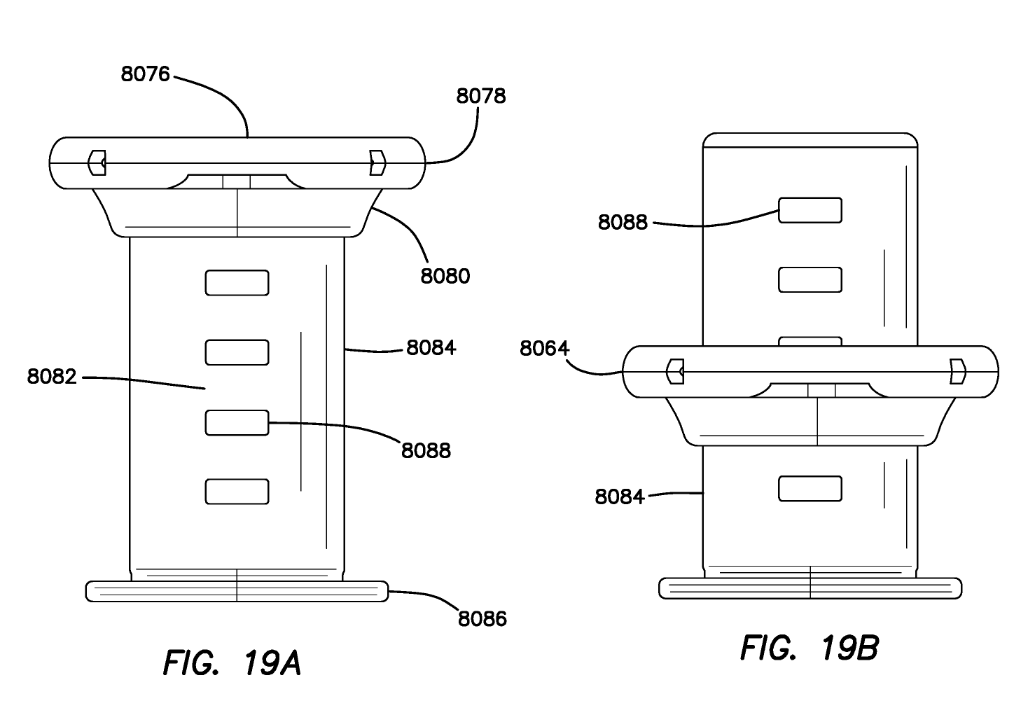

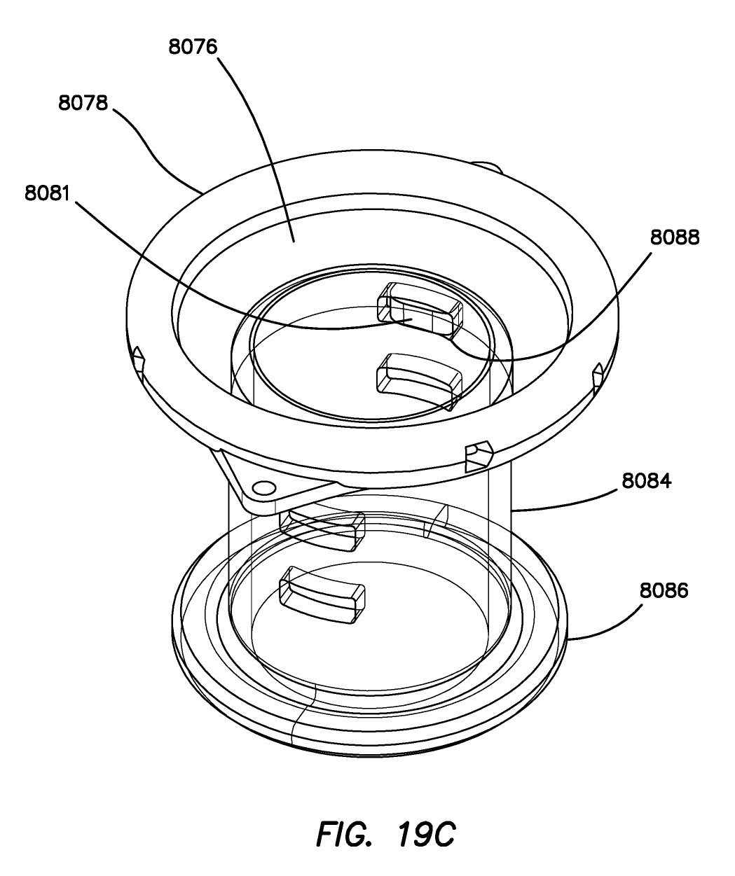

Another embodiment of the two-piece access device is shown in FIGS. 19A-C. In this embodiment, the exterior portion 8076 comprises an outer ring 8078 and a funnel portion 8080. One or more tabs 8081 protrude inwardly from the inner surface of the funnel portion (shown in FIG. 19C). The interior portion 8082 comprises a tubular body 8084 and an inner ring 8086, with two or more slots 8088 disposed through the tubular body, adapted to receive the tab 8081.

To adjust the length of the access device, the user slides the exterior portion 8076 along the outer surface of the tubular body 8084 of the interior portion until the desired distance between the inner ring 8086 and outer ring 8078 is achieved (as shown in FIG. 19B). The exterior portion is rotated to matingly engage the tab 8081 into the cut-out 8088 and excess tubular body extending beyond the outer ring is removed using a scalpel or other sharp instrument. Alternatively, the desired length of the access device can be approximated before disposing the exterior portion around the interior portion. Excess tubular body can be excised prior to attaching the exterior portion, taking care to leave the appropriate cut-out intact to receive the tab. The assembled access device, cut to appropriate length, is shown in FIG. 19C.

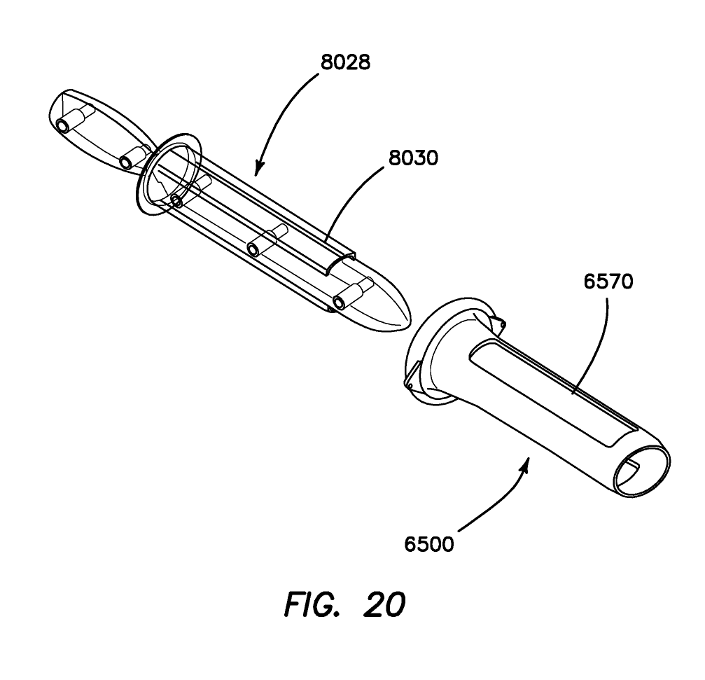

As described above, the access device of the natural orifice system can be modified in several described embodiments to provide for adjustments in length, both to facilitate access to various portions of the natural orifice and to accommodate patients having different body types and BMI. In addition, as shown in FIGS. 7C-7F and FIG. 20, the tubular body of the access device can optionally include cut-out portions or windows 6570, to provide access to regions of the anatomy that would otherwise be obscured by the tubular body while the access device is in place. Thus, the access device can be inserted into the body orifice or incision to provide retraction and to protect the lining of the body cavity, and then manipulated to align the window(s) to the sites of interest in the body cavity for access by surgical instruments.

As discussed above, an obturator may be used to place the access devices of the present invention into the natural orifice of the patient. An obturator adapted for use with an access device including cut-out portions in the tubular body is shown is FIG. 20. This obturator 8028 is similar to that described in FIG. 6I, but further comprises one or more raised flanges 8030, adapted to interface with the cut-out potions 6570 of the access device 6500. When the obturator is inserted into the access device such that the flange(s) is mated with the cut-out portion(s), the flange ensures a smooth and consistent contact surface and facilitates rotating the access device to ensure the cut-out portion is aligned with the surgical site.

As will be appreciated, such cut-out portions may be provided in access devices having tubular bodies of both rigid and flexible construction, as well as tubular bodies formed as a single piece or in sections. FIG. 7M shows an example of a flexible tubular body of an access device, both in side view and in perspective view, wherein the tubular body contains perforations 6580. The tubular body can be cut or torn at the perforations to vary the length of the tubular body and/or to incorporate cut-out portions into the tubular body. The tubular body of the embodiment shown in FIG. 7M is preferably formed from a relatively flexible material, such as KRATON® polymer or PELLETHANE® thermoplastic polyurethanes.

In the illustrated embodiments of FIGS. 9A and 9B, the trocar access devices 6310 have a relatively low profile, that is, protrude minimally above the access surface 6320 and/or below the distal surface of the cap 6300. Accordingly, the trocar access devices 6310 are shorter than a length of a typical trocar and comprise a seal assembly positioned above the access surface 6320 and a cannula extending through the gel pad of the cap 6300. The reduced length of the trocar access devices 6310 allows increased angular or pivotal motion for instruments extending there through, and also permits the use of curved and/or angled instruments.

FIG. 9C is an exploded view of an embodiment of a trocar access device 6310 and optional obturator 6600, which is a component of some embodiments of the access device system. In the illustrated embodiment, the obturator 6600 comprises a pointed, puncture tip 6610.

The trocar access device 6310 comprises a proximal end, a distal end, and a longitudinal axis. The trocar access device 6310 comprises a cannula 6620 extending along the longitudinal axis. A trocar seal 6630 is disposed at the proximal end of the cannula 6620, contained within a housing 6640. A retainer 6650 is disposed at the distal end or tip of the cannula 6620.

The cannula 6620 comprises a tubular body dimensioned to accommodate an instrument or instruments received there through. In the illustrated embodiment, the cannula 6620 is a substantially cylindrical tube, and extends through the cap 6300 in use. In the illustrated embodiment, the cannula 6620 is comparatively short because the cannula need only traverse the cap 6300 (FIG. 9A-B), which has a known and consistent thickness, rather than a body wall. Accordingly, some embodiments of the cannula 6620 are not more than about 2-times longer, about 1.5-times longer, about 1.2-times longer, or about 1.1-times longer than the thickness of the gel pad. In some embodiments, the cannula 6620 is less than about 20 mm, about 10 mm, or about 5 mm longer than the thickness of the gel pad. In some embodiments, the cannula 6620 is about as long as the gel pad is thick. In other embodiments, the cannula 6620 has a different length, for example, a length typical for a cannula used for traversing a body wall. Shorter length cannula permit increased angular degrees of freedom for instruments passing there through. Embodiments of shorter cannula also accommodate curved instruments. The cannula 6620 comprises any suitable biocompatible material. In some embodiments, the cannula 6620 comprises a flexible material.

The illustrated trocar seal 6630 comprises an instrument or septum seal 6660 and a zero seal 6670. Optionally, a shield 6680 may be disposed within the instrument seal 6660. The instrument seal 6660 seals instruments passing there through, thereby maintaining pressurization in a body cavity such as pneumoperitoneum or pneumorectum. The zero seal 6670 provides a seal when no instrument passes through the trocar seal 6630. The instrument seal 6660 and zero seal 6670 are received in a housing 6640 disposed at the proximal end of the cannula 6620 and secured therein by a seal cover 6690.

The retainer 6650 is disposed at or near the distal end of the cannula 6620. In some embodiments, the retainer 6650 and cannula 6630 are integrated, while in other embodiments, the retainer 6650 and cannula 6630 are not integrated. In the illustrated embodiment, the proximal end of the retainer 6650 comprises a flange 6655 that is generally flat and perpendicular to the longitudinal axis, while the distal end is tapered, narrowing toward the distal end of the cannula 6620. The flange 6655 reduces the likelihood of accidental or inadvertent removal of the trocar access device 6310 from the cap. Some embodiments of the proximal face of the flange 6655 comprise additional anchoring features, for example, at least one of barbs, spikes, ridges, texturing, and the like, which are configured to penetrate or bite into a distal face of the cap 6300. In some embodiments, a diameter of the flange 6655 is from about 1.2 to about 2.5 times wider, or from about 1.5 to about 2.0 times wider than an outer diameter of the cannula 6630. Some embodiments of the trocar access device 6310 are 5-mm trocars, in which the outer diameter of the cannula 6620 is from about 7 mm to about 8 mm.

The tapered end of the retainer 6650 facilitates insertion of the trocar access device 6310 through the cap, either by itself, or when assembled with the obturator 6600 extending there through. For example, in some embodiments, the retainer 6650 is inserted through a preformed opening in the cap 6300.

In some embodiments in which the retainer 6650 and cannula 6620 are not integrated, that is, are separate components, the retainer 6650 is secured to the cannula 6620 after the cannula 6620 is inserted through the cap. In some embodiments, the cannula 6620 and retainer 6650 are secured mechanically, for example, using latches, screw threads, clips, lock rings, ratchets, and the like. In some embodiments, the cannula 6620 and retainer 6650 are secured adhesively. In some embodiments, the position of the retainer 6650 is adjustable, for example, to accommodate caps of different thicknesses. In some embodiments, the cannula 6620 and/or retainer 6650 is secured to the cap, for example, adhesively.

An embodiment of a procedure for retracting a body orifice is described with reference to the embodiments of the access device 6100 illustrated in FIGS. 6A-6C, 7A, 8A-8C, and 9A, and the embodiments of access device 7100 illustrated in FIGS. 6D-6F, 7B, 8D, and 9B, although the procedure is applicable to all of the embodiments of the access device disclosed herein. In use, the natural orifice access device 6100, 7100 is inserted into a body orifice, such as the vagina (FIG. 2), mouth (FIG. 3) or anus (FIG. 4). The inner ring 6110, 7110 is folded or compressed into an oval or other suitable shape and urged through the incision or body orifice into an associated body cavity. Once the inner ring 6110, 7110 is fully disposed within the associated body cavity, it is allowed to resume its original, relaxed shape, for example, substantially circular, oval, or other original shape. In some embodiments, the inner ring 6110 is then pulled upward against the inner surface of the body cavity, for example, by pulling the outer ring 6120 upward. An outer surface of the tubular body 6130, 7130 retracts the natural orifice.

As illustrated in FIG. 5, some embodiments of the access device system 5000 comprise a cap, cover, or lid 5500 coupled to the outer ring of the access device 5100, which seals the access device 5100, for example, for maintaining pressurization within a body cavity such as pneumoperitoneum or pneumorectum. In some embodiments, lid 5500 is removable, for example to provide access into the body cavity. Some embodiments of the lid 5500 comprise a transparent or translucent portion, thereby allowing a user to view into the body cavity without removing the lid 5500. As will be described below, one embodiment of a lid 5500 is a gel cap. In some embodiments, a cross-sectional shape of the outer ring 6120 (FIG. 6A), 7120 (FIG. 6D) of the access device is selected to reduce or prevent the lid 5500 from partial and/or incorrect coupling to the outer ring 6120 (FIG. 6A), 7120 (FIG. 6D) of the access device. Such cross-sectional shapes include oval and rectangular, or any other suitable cross-sectional shape that provides the desired functionality, for example, hexagonal, octagonal, and the like. Additionally, depending on the use and on surgeon preference, in some embodiments, each of the inner ring 6110, 7110 and outer ring 6120, 7120 of the access device includes independently variable design configurations. For example, embodiments of the inner ring 6110, 7110 and/or the outer ring 6120, 7120 are rigid or flexible, and have footprints, cross-sectional shapes, and/or dimensions dependent on the intended use, for example, circular or oval footprints, diameters dependent on incision or orifice dimensions, or cross-sectional dimensions dependent on retraction force. In some embodiments, the inner ring 6100 may extend radially out from the tubular body 6130 when deployed, stabilizing the access device within the body orifice (FIG. 7A). In other embodiments, the inner ring 7110 may be flush with the tubular body 7130, as where, for example, the length L2 of the tubular body is sufficient to stabilize the access device within the body orifice (FIG. 7B).

Various embodiments of a natural orifice surgery system useful in anal, vaginal, and oral procedures have been described. These embodiments are particularly useful for providing variable access to portions of a natural orifice by varying the length of the access channel. These embodiments are also useful in patients having higher BMI, as the adjustability of the channel will facilitate anchoring of the device in patients of different body types. While certain embodiments have been particularly shown and described with reference to exemplary embodiments thereof, it will be understood by those of ordinary skill in the art that various changes in form and details may be made therein without departing from the spirit and scope thereof as defined by the following claims.

What is claimed is:

1. A surgical access system adapted for performing laparoscopic surgical procedures at a natural orifice comprising:
an outer ring, wherein the outer ring is configured to be disposed proximate the natural orifice of the patient and surround the orifice;
a tubular body having a proximal end, a distal end, a longitudinal axis and a cut-out portion disposed through a side of the tubular body;
a funnel segment extending between and coupling the outer ring and the proximal end of the tubular body, wherein the funnel segment comprises an inner surface that can provide a bearing surface for an obturator used to advance to the retractor into a body cavity and provides a diametric reduction between the larger diameter of the outer ring and the smaller diameter of the tubular body, which is sized to fit within a natural orifice with minimal distention of the orifice; and
an obturator comprising a proximal bearing surface sized and configured to bear against the inner surface of the funnel segment, a distal dilation surface sized and configured to expand the natural orifice, a straight shaft piece disposed between the distal dilation surface and the proximal bearing surface, and a raised flange disposed on the straight shaft piece, adapted to interface with the cut-out potion to provide a smooth and consistent contact surface during insertion.

2. The surgical access device of claim 1, wherein the tubular body comprises a flexible material.

3. The surgical access device of claim 1, wherein the tubular body comprises a polycarbonate.

4. The surgical access device of claim 1, wherein the tubular body comprises one or more coatings.

5. The surgical access device of claim 4, wherein the coating comprises an anti-microbial coating.

6. The surgical access device of claim 1, wherein the tubular body is formed from a flexible material and the funnel segment and the outer ring are formed from a rigid material.

7. The surgical access device of claim 1, further comprising at least one suture point disposed on the exterior portion adjacent the outer ring.

8. The surgical access device of claim 1, further comprising a removable cap, wherein the cap is adapted to sealingly engage the outer ring.

9. The surgical access device of claim 8, wherein the cap comprises a sealable access surface.

10. The surgical access device of claim 9, wherein the sealable access surface is a gel pad.
